(19) United States
(12) Patent Application Publication
(10) Pub. No.: US 2016/0310604 A1
(43) Pub. Date: Oct. 27, 2016

(54) SMALL MOLECULE LIGAND-DRUG CONJUGATES FOR TARGETED CANCER THERAPY

(71) Applicants: Cedars-Sinai Medical Center, Los Angeles, CA (US); Emory University, Atlanta, GA (US); Georgia State University Research Foundation, Inc., Atlanta, GA (US)

(72) Inventors: Leland W.K. Chung, Beverly Hills, CA (US); Xiaojian Yang, Shannxi (CN); Jianjun Cheng, Champaign, IL (US); Rong Tong, Urbana, IL (US); Maged Henary, Lawrenceville, GA (US)

(21) Appl. No.: 15/006,734

(22) Filed: Jan. 26, 2016

Related U.S. Application Data
(63) Continuation of application No. 12/995,417, filed on Nov. 30, 2010, now abandoned.
(60) Provisional application No. 61/061,346, filed on Jun. 13, 2008.

(51) Int. Cl. A61K 47/48; A61K 49/00

(52) U.S. Cl. CPC A61K 47/48061; A61K 49/0002; A61K 49/0052; A61K 49/0032

(57) ABSTRACT

The present invention describes small molecule ligand-drug conjugates and methods of using the small molecule ligand-drug conjugates for targeted treatment of cancer in a patient in need thereof. Further described are methods of sterilizing circulating tumor cells and determining drug concentration in cancer tissue.

### STATEMENT REGARDING FEDERALLY SPONSORED RESEARCH OR DEVELOPMENT

This invention was made with government support under Grant number 0748834 awarded by the National Science Foundation and Grant number CA-119338 from the National Cancer Institute. The government has certain rights in the invention.

### BACKGROUND OF THE INVENTION

Cancer is the second leading cause of death in the US. Most deaths from cancer are caused by metastasis for which there is no effective therapy. Improved drug delivery to cancer cells is critical for the development of effective chemotherapy in patients. One approach is to synthesize chemical conjugates of promising drugs with a targeting ligand that recognizes a unique biomarker on the surface of a cancer cell. Unfortunately, because of the heterogeneity and evolutional properties of cancer cell surface biomarkers and the bulky chemical structures of the targeting ligands, this type of targeting approach, although promising, remains a challenge from both technical and translational points of view.

There is a need for an improved drug delivery system specific for cancer having time-dependent release of the drug payload where the released drug is capable of inducing maximal cancer cell-kill but causes little or no damage to the normal host cells.

### SUMMARY OF THE INVENTION

The present invention provides a small molecule conjugate compound comprising: a targeting ligand; a therapeutic agent and/or an imaging agent; and a linker connecting the ligand to the therapeutic agent and/or the imaging agent.

In certain embodiments, the targeting ligand comprises an electron withdrawing group or an electron donating group.

In other embodiments, the targeting ligand comprises: an indole portion; a polyen portion; and a side chain portion.

In certain embodiments, the indole portion, the polyen portion and/or the side chain portion comprises a conjugation amenable functional group. In various embodiments, the conjugation amenable functional group may be selected from the group consisting of OH, NH₂, SH, and COOH.

In certain embodiments, the indole portion and the polyen portion are represented by the following formula:

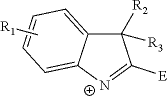

wherein E represents the polyen portion and R₁, R₂, and R₃ may each be independently selected from the group consisting of: OH; NH₂; SH; COOH; H; C1-C15 alkyl and may be optionally substituted with one or more nitrogen-containing groups, oxygen-containing groups, sulfur-containing or halogen atoms; alkoxy and may be optionally substituted with one or more nitrogen-containing groups, oxygen-containing groups, sulfur-containing or halogen atoms; aryl and may be optionally substituted by one or more heteroatoms or substituents; aromatic ring and may be optionally substituted by one or more heteroatoms or substituents; non-aromatic ring and may be optionally substituted by one or more heteroatoms or substituents; oxy; carbonyl; alkenyl; nitro; and amino.

In certain embodiments, the polyen portion may be a polyen substituted with a substituent selected from the group consisting of OH, NH₂, SH, and COOH.

In certain embodiments, the polyen portion may be a dien, trien or tetraen and may be optionally substituted with one or more heteroatoms or substituents; optionally contains an aryl that may be optionally substituted by one or more heteroatoms or substituents; optionally contains an aromatic ring that may be optionally substituted by one or more heteroatoms or substituents; or optionally contains a non-aromatic ring that may be optionally substituted by one or more heteroatoms or substituents, wherein the one or more substituents may be selected from the group consisting of OH, NH₂, SH, and COOH.

In certain embodiments, the side chain portion and the indole portion is represented by the following formula:

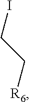

wherein I represents the indole portion and R₆ may be selected from the group consisting of: OH; NH₂; SH; COOH; H; C1-C15 alkyl and may be optionally substituted with one or more nitrogen-containing groups, oxygen-containing groups, sulfur-containing or halogen atoms; alkoxy and may be optionally substituted with one or more nitrogen-containing groups, oxygen-containing groups, sulfur-containing or halogen atoms; aryl and may be optionally substituted by one or more heteroatoms or substituents; aromatic ring and may be optionally substituted by one or more heteroatoms or substituents; non-aromatic ring and may be optionally substituted by one or more heteroatoms or substituents; oxy; carbonyl; alkenyl; nitro; and amino.

In certain embodiments, the indole portion may be selected from the group consisting of:

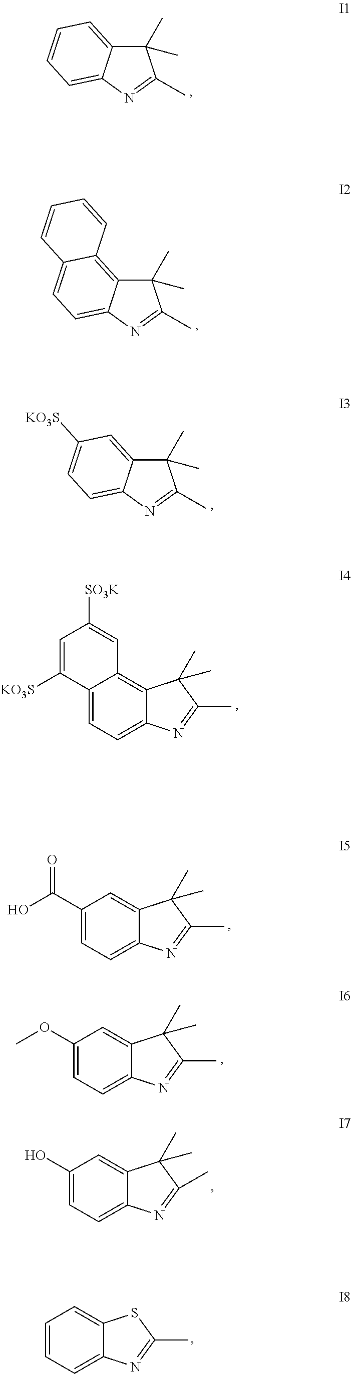

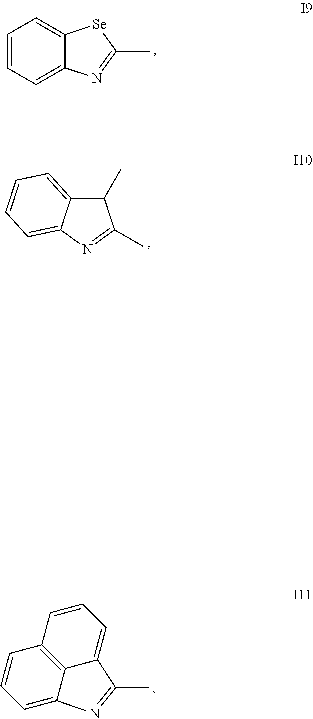

and combinations thereof.

In certain embodiments, the polyen portion and the indole portion is selected from the group consisting of:

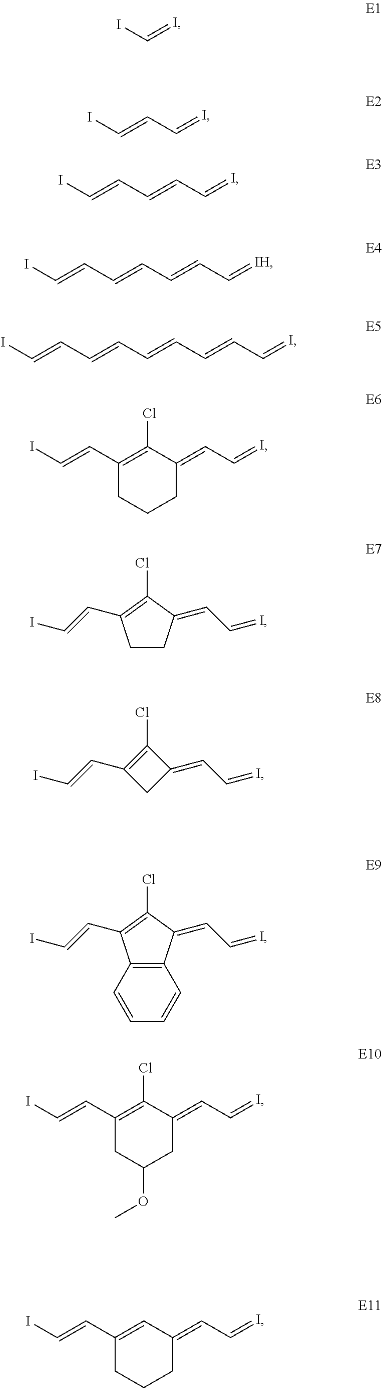

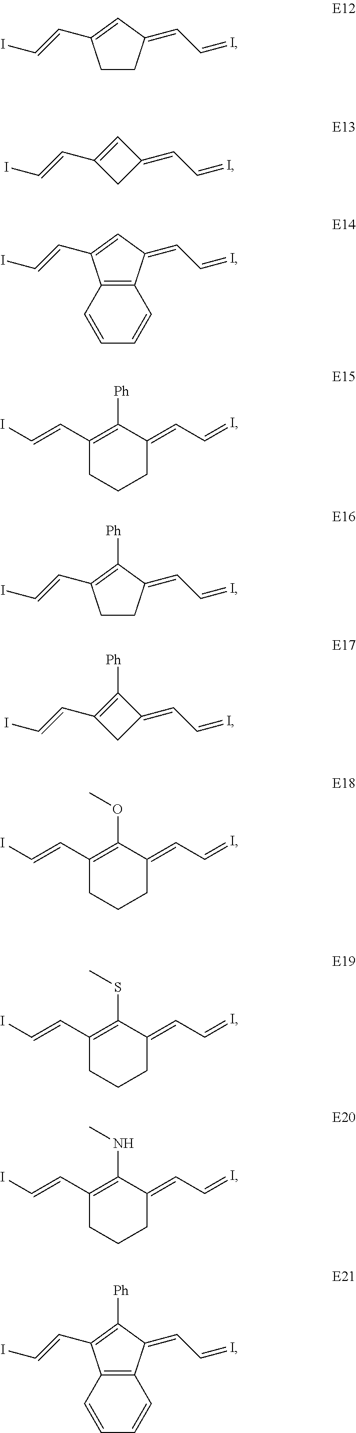

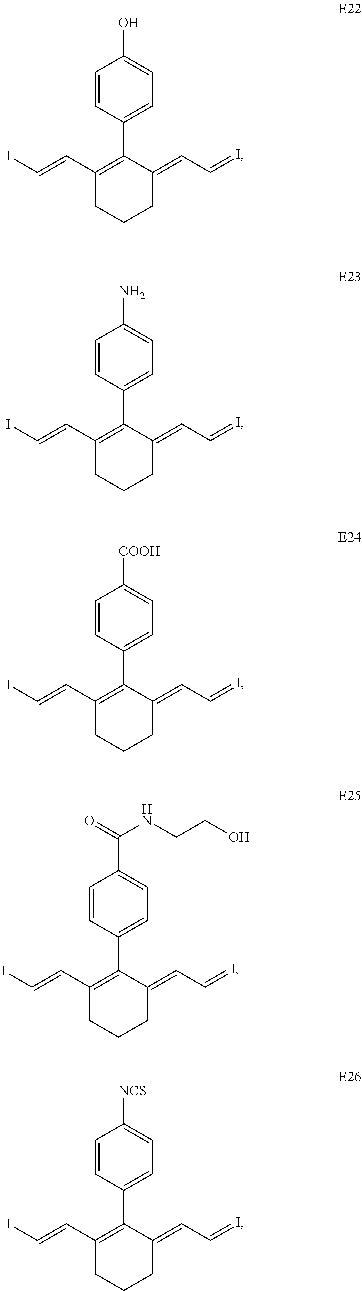

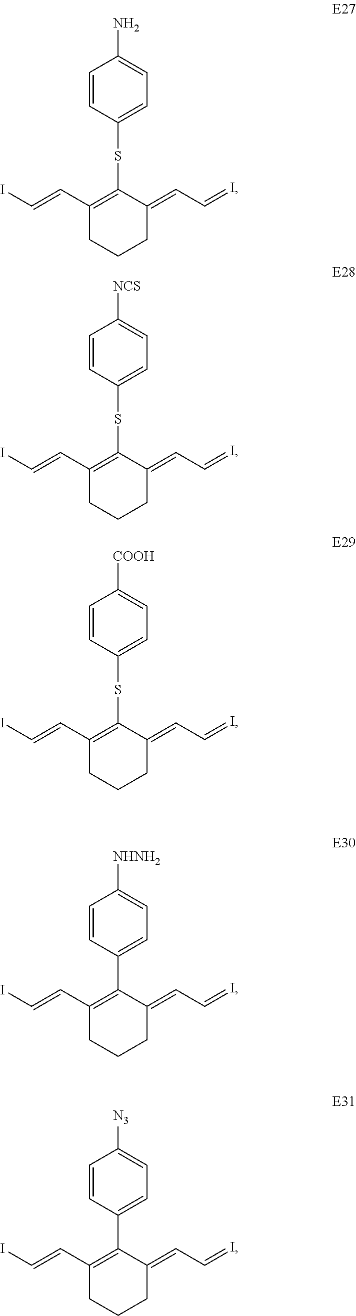

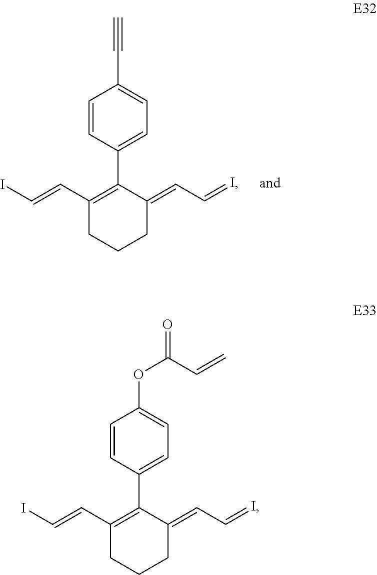

wherein the I represents the indole portion of the compound.

In certain embodiments, the side chain portion and the indole portion is selected from the group consisting of:

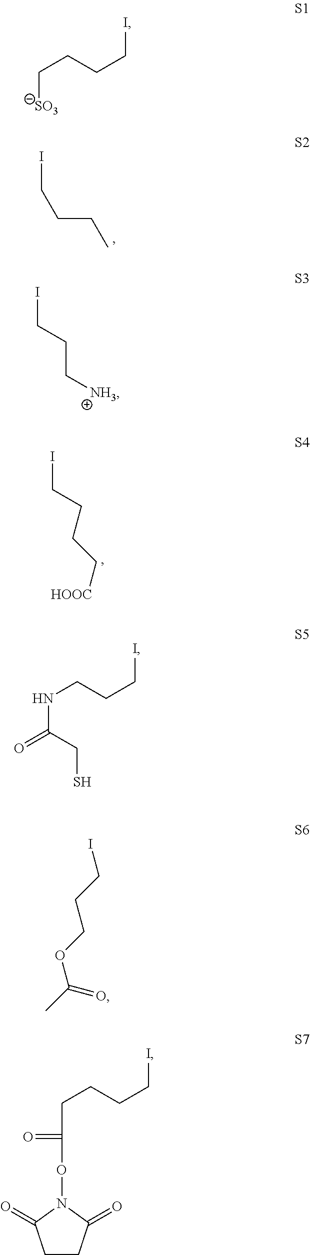

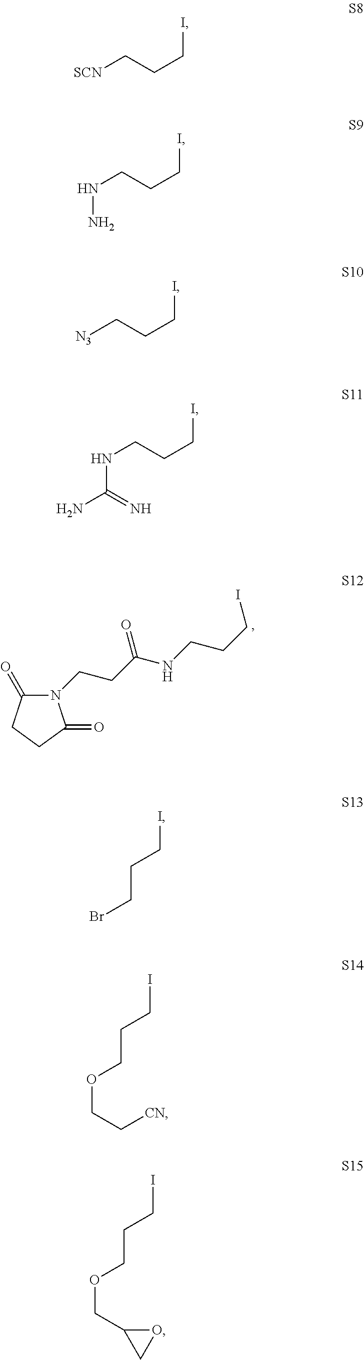

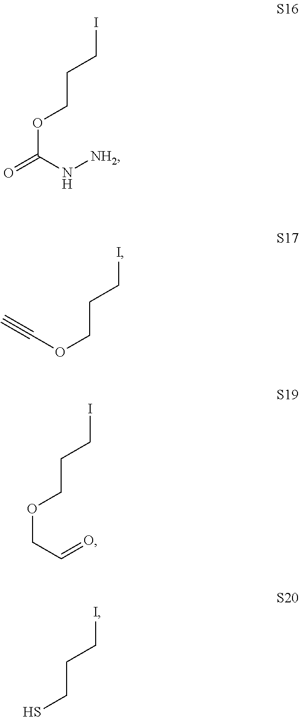

and combinations thereof, and wherein the I represents the indole portion.

In certain embodiments, the targeting ligand is a polyen connecting two aliphatic indoles.

In certain embodiments, the polyen may contain two to four conjugated double bonds.

In certain embodiments, the targeting ligand may be a cyanine dye. In various embodiments, the cyanine dye may be represented by the following formula:

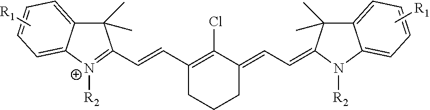

wherein R₁ and R₂ are each independently selected from the group consisting of: H; C1-C15 alkyl and may be optionally substituted with one or more nitrogen-containing groups, oxygen-containing groups, sulfur-containing or halogen atoms; alkoxy and may be optionally substituted with one or more nitrogen-containing groups, oxygen-containing groups, sulfur-containing or halogen atoms; aryl and may be optionally substituted by one or more heteroatoms or substituents; aromatic ring and may be optionally substituted by one or more heteroatoms or substituents; non-aromatic ring and may be optionally substituted by one or more heteroatoms or substituents; oxy; carbonyl; alkenyl; nitro; and amino.

In other embodiments, the cyanine dye may be selected from the group consisting of:

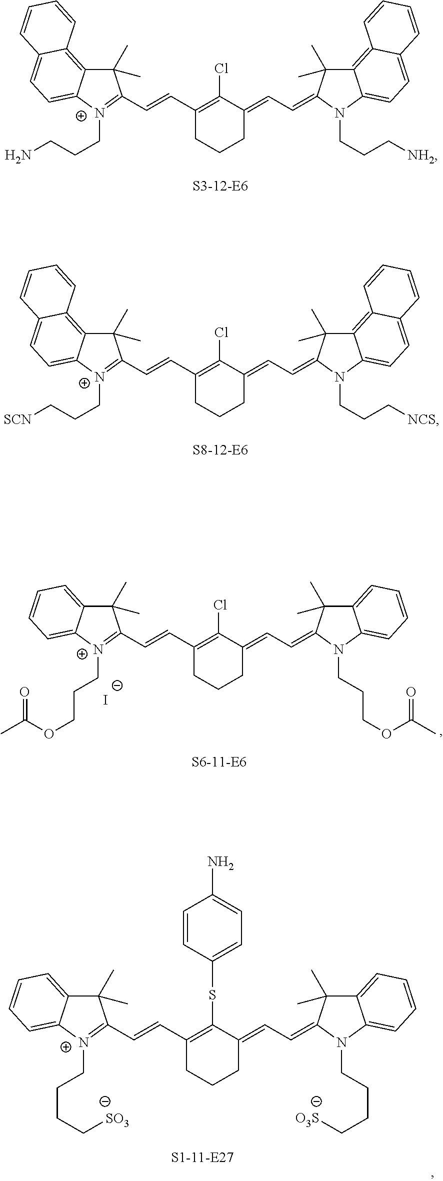

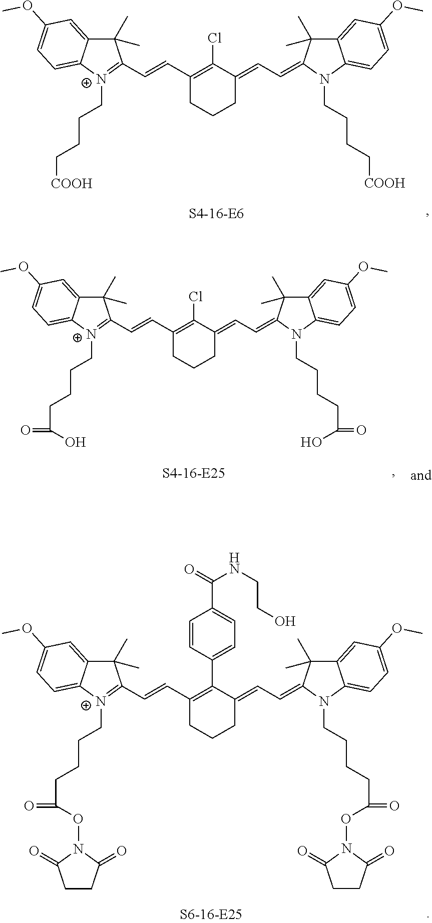

In certain embodiments, the targeting ligand may be IR-783 or a derivative thereof. In various embodiments, the IR-783 derivative may be selected from the group consisting of: S2-I3-E2, S4c-I1-E4cCI, S1-I2-E3, S1-I4-E3CI, S1-I1-E3, S5c-I1-E4cCI, S5-I1-E4cCI, S3-I1-E4cCI, S4s-I1-E4cba, S3p-I1-E4cCI, S4ac-I1-E4cCI, S3-I1-E3, and S2-I1-E4cCI.

In certain embodiments, the targeting ligand may be a dye having wavelength of maximum fluorescence emission greater than 700 nm.

In certain embodiments, the linker may be selected from the group consisting of: succinic ester, amino acid, peptide, diacid, bisamine, bis-alcohol, anhydride, CN, an alkyne group capable of a click reaction, epoxy, hydrazine, azide, aldehyde, ketone, sulfonic acid, phosphoric acid, phosphoamidite, guanidine, short (C1-C6) alkyl, aromatic group, ester, amide, urea, thiourea, imidazole, imidazole derivative, thioester, acrylate, thiol ether, dithioate, selenide and phenyl selenide, diene, diketone, pyrimidine, purine, heterocyclic ring structure, crown ether, phenoldiazene, nitrobenzene, nitrobenzene derivative, iodo or bromo, monosaccharide, oligosaccharide, azirine, benzophenone, bipyridine, biphenol, aminophenol, indole derivative capable of acting as an electrochemical crosslinker, radioactive atom, chelator for a radioactive atom and combinations thereof.

In certain embodiments, the therapeutic agent may be selected from the group consisting of: anti-cancer drug capable of targeting cell growth, survival, angiogenesis, adhesion, migration, invasion, metastasis, cell cycle progression and/or cell differentiation; small molecule drug capable of targeting cell growth, survival, angiogenesis, adhesion, migration, invasion, metastasis, cell cycle progression and/or cell differentiation; bisphosphonate drug for metastatic bone cancer treatment; peptide therapeutic agent and combinations thereof.

In certain embodiments, the composition may further comprise a ligand capable of recognizing tumor stroma, tumor cells, and/or matrices in a tumor microenvironment. In various embodiments, these ligands may be arginine-glycine-aspartic acid (“RGD”) peptide recognizing cell surface integrin receptors, growth factors such as EGF, PDGF, VEGF recognizing cell surface growth factor receptors, peptides or small molecule substrates that recognize functional cell surface plasminogen activator, bombesin, bradykinin or prostate specific membrane antigen receptors.

In certain embodiments, the anti-cancer drug may be selected from the group consisting of: aminoglutethimide, asparaginase, bleomycin, busulfan, carboplatin, carmustine (BCNU), chlorambucil, cisplatin (cis-DDP), cyclophosphamide, cytarabine HCl, dacarbazine, dactinomycin, daunorubicin HCl, doxorubicin HCl, estramustine phosphate sodium, etoposide (VP-16), floxuridine, fluorouracil (5-FU), flutamide, hydroxyurea, hydroxycarbamide, ifosfamide, interferon a-2a, interferon a-2b, leuprolide acetate, lomustine (CCNU), mechlorethamine HCl, melphatan, mercaptopurine, mesna, methotrexate (MTX), mitomycin, mitotane (o.p′-DDD), mitoxantrone HCl, octreotide, plicamycin, procarbazine HCl, streptozocin, tamoxifen citrate, thioguanine, thiotepa, vinblastine sulfate, vincrinstine sulfate, amsacrine (m-AMSA), azacitidine, hexamethylmelamine (HMM), interleukin 2, mitoguazone (methyl-GAG, methyl glyoxal bis-guanylhydrazone (MGBG)), pentostatin, semustine (methyl-CCNU), teniposide (VM-26), paclitaxel, docetaxel, taxane, vindesine, and sulfate.

In particular embodiments, the therapeutic agent may be paclitaxel or docetaxel.

In certain embodiments, the small molecule drug may be selected from the group consisting of antibody, antisense nucleic acid, small interference RNA, and micro RNA. In certain embodiments, the bisphosphonate drug may be zolendrate or palmedranate. In certain embodiments, the peptide therapeutic agent may be cyclosporine or samatostatin.

In certain embodiments, the compound may be S4s-I1-E4cCI-Suc-docetaxel or S4s-I1-E4cCI-Suc-paclitaxel.

In certain embodiments, therapeutic agent may be an alpha emitter. In various embodiments, wherein the alpha emitter may be radium-223, uranium-238, thorium-232, polonium-210, or actinium-225.

In certain embodiments, the imaging agent may be a positron emission tomography (PET) imaging agent or a magnetic resonance imaging (MRI) contrasting agent. In various embodiments, the PET imaging agent may be fluorine-18 (F-18), carbon-11 (C-11), nitrogen-13 (N-13), or oxygen-15 (O-15). In a particular embodiment, the MRI contrasting agent may be gadolinium.

The present invention also provides a method of treating cancer in a patient in need thereof, comprising: providing a small molecule conjugate compound of the present invention; and administering an effective amount of the compound to the patient.

The present invention also provides a method of sterilizing circulating tumor cells in a patient in need thereof comprising: providing a small molecule conjugate compound of the present invention; and administering an effective amount of the compound to the patient, wherein subsequent adhesion and/or extravasations of a cancer cell to form a metastatic deposit are minimized or prevented.

The present invention also provides a method of determining drug concentration in cancer tissue, comprising: providing a small molecule conjugate compound of the present invention; administering an effective amount of the compound to a patient in need thereof or to a tissue; and imaging the patient or tissue; and correlating the intensity of the image with the amount of drug in the tissue.

The present invention also provides a method of imaging a cancer cell or cancer tissue, comprising: providing a small molecule conjugate compound of the present invention; administering an effective amount of the compound to a patient in need thereof or to a tissue, wherein the imaging agent is a magnetic resonance imaging (MRI) contrasting agent or a positron emission tomography (PET) imaging agent; and imaging the patient or tissue.

### BRIEF DESCRIPTION OF THE FIGURES

FIG. 1A depicts one representation of a cyanine-dye conjugate developed for targeting cancer therapy in accordance with an embodiment of the present invention.

FIG. 1B depicts the chemical structure of S4s-I1-E4cCI-Suc-Dtxl in accordance with an embodiment of the present invention.

FIG. 2A and FIG. 2B depict mass spectra of S4s-I1-E4cCI-Suc and S4s-I1-E4cCI-Suc-Dtxl in accordance with an embodiment of the present invention.

FIGS. 3A-3F are photographs depicting the uptake of S4s-I1-E4cCI and S4s-I1-E4cCI-Suc-Dtxl in SN12C cells in accordance with an embodiment of the present invention. S4s-I1-E4cCI (20 uM) was incubated with SN12C cells (a human renal cancer cell line) for 30 minutes at 37° C., washed and subjected to confocal imaging (FIG. 3A). Fluorescence images were recorded on a fluorescent microscope (ZEISS® LSM 510 META, Germany) equipped with a 703 nm laser (λₑₓ=800 nm and λₑₘ=850 nm); a bright-field image of the cells imaged in FIG. 3A is shown in FIG. 3B; and FIG. 3C is an overlay of FIG. 3A and FIG. 3B. The uptake experiment of S4s-I1-E4cCI-Suc-Dtxl was performed similarly (FIGS. 3D-3F). IR-783-Suc-docetaxel was incubated with SN12C cells for 30 minutes at 37° C. Fluorescence images were taken at λₑₓ=800 nm and λₑₘ=850 nm (FIG. 3D); a bright-field image of the cells imaged in FIG. 3D is shown in FIG. 3E; and FIG. 3F is an overlay of FIG. 3D and FIG. 3E.

FIGS. 4A-4C are photographs depicting the in vivo targeting of S4s-I1E4cCI-Suc-Dtxl in accordance with an embodiment of the present invention. Whole-body NIR optical imaging and X-ray of athymic nude mice with subcutaneously implanted human bladder cancer T24 cells 48H after intravenous injection of S4s-I1-E4cCI-Suc-Dtxl is shown in FIG. 4A. Experimental condition: 1×10⁶ human bladder cancer T24 cells were subcutaneously injected into athymic nude mice at both flanks of the animal. After tumor sizes reached approximately 7-8 mm³ in diameter, mice were injected intravenously into tail vein with S4s-I1-E4cCI-Suc-Dtxl at a dose of 10 nmol per mouse. Whole-body NIR optical imaging and X-ray of the animals were conducted on a KODAK® In Vivo Animal Imaging Station (New Haven, Conn.) equipped with 800 nm filter sets (excitation/emission, 800/850 nm). Images were analyzed using KODAK® ID3.6.3 network version imaging. The fluorescence intensity can be achieved above 500 arbitrary unit; a bright field image of the same mouse shown in FIG. 4A is shown in FIG. 4B; and FIG. 4C is an overlay of FIG. 4A and FIG. 4B.

FIG. 5 depicts a time course study of in vivo cancer targeting and retention of S4s-I1-E4cCI-Suc-Dtxl in accordance with an embodiment of the present invention.

FIG. 6 shows that IR-MUT1 is toxic and can kill cancer cells, but is less toxic than the free nonconjugated drug when evaluated at day 2 in accordance with an embodiment of the present invention.

FIG. 7A and FIG. 7B are photographs depicting an assessment of apoptosis of mouse tumor tissue in accordance with an embodiment of the present invention. FIG. 7A shows T24 human bladder tumor xenograft nude mouse treated with IR-MUT1. Note that tumor apoptosis can be seen in IR-MUT1-treated specimen. FIG. 7B shows T24 human bladder xenograft mouse without treatment.

FIG. 8 depicts the naming scheme for dye molecules of the invention in accordance with an embodiment of the present invention. The suggested name of IR783 (MUT) series dye is S4h-I1-E4cCI. S: side chain; 4: 4CH₂; h (lowercase): hydroxyl (amine (a), COOH (c), acetate (ac), SO₃₋ (s), ph (p)); I: indole; E: polyen; 4: 4 en; c (lower case): cyclo; Cl: chloride (Cl).

FIG. 9 depicts drug conjugates with mono-, di- and tri-functional dye molecules in accordance with an embodiment of the present invention.

FIG. 10 depicts an in vitro study showing the active uptake of IR-MUT1 by human renal cancer cells but not normal human fetal kidney cells in culture in accordance with an embodiment of the present invention. Renal cancer cells (1×10⁴/well) and normal cells were seeded on vitronectin-coated four-well chamber slides. IR-MUT1 was added at a concentration of 20 μM. The slides were incubated at 37° C. for 30 min and then fixed with 10% formaldehyde at 4° C. Images were recorded by confocal laser microscopy (ZEISS® LSM 510 META, Germany) equipped with 633 nm laser and 650 nm fluorescent filters. Significant uptake of IR-MUT1 by renal cancer cells (SN12C, ACHN and Caki-1) was observed. In contrast, the uptake of IR-MUT1 by normal human fetal kidney cells (HEK293) was marginal to undetectable.

FIG. 11 depicts another in vitro study showing active uptake of IR-MUT1 by human prostate cancer but not normal human prostate epithelial cells in culture in accordance with an embodiment of the present invention. Prostate cancer cells (1×10⁴/well of C4-2, PC3, ARCaP-M and ARCaP-E) and normal prostate epithelial cells (1×10⁴/well of P-69) were seeded on vitronectin-coated four-well chamber slides. IR-MUT1 was added at a concentration of 20 μM. The slides were incubated at 37° C. for 30 min and then fixed with 10% formaldehyde at 4° C. Images were recorded by confocal laser microscopy (ZEISS® LSM 510 META, Germany) equipped with 633 nm laser and 650 nm fluorescent filters. Significant uptake of IR-MUT1 by prostate cancer cells (C4-2, PC3, ARCaP-M, ARCaP-E) was observed. While the uptake of IR-MUT1 by human epithelia prostate cells (P69) was marginal to undetectable.

FIG. 12 depicts another in vitro study showing active uptake of IR-MUT1 by both human and mouse pancreatic cancer cells in culture in accordance with an embodiment of the present invention. Pancreatic cancer cells (1×10⁴/well) were seeded on vitronectin-coated four-well chamber slides. IR-MUT1 was added at a concentration of 20 μM. The slides were incubated at 37° C. for 30 min and then fixed with 10% formaldehyde at 4° C. Images were recorded by a confocal laser microscope (ZEISS®LSM 510 META, Germany) equipped with 633 nm laser and 650 nm fluorescent filters. Significant uptake of IR-MUT1 by human pancreatic cancer cells (MIA PACA2, BXPC3) and mouse pancreatic cancer cells (PDAC2.3) was observed.

The graphs of FIG. 13A, FIG. 13B, FIG. 13C, and FIG. 13D depict another in vitro study showing IR-MUT1 inhibited greater human prostate cancer cell (C4-2) growth than those of the normal human prostate epithelial (P-69) cells in vitro in accordance with an embodiment of the present invention. C4-2 (FIG. 13A) and P69 (FIG. 13B) cells were plated in 96 well plates (3,000/well). After attachment overnight, the cells were incubated with IR-MUT1 for 48 hrs. The MTT assay was employed to determine and compare the cytotoxicity of IR-MUT1 in C4-2 and P69 cells grown in vitro. The figure showed IR-MUT1 inhibited human prostate cancer cell growth in culture with an identical IC50 of 10 nM as that of Taxotere. In contrast, the cytotoxicity rendered by IR-MUT1 in P69 cells is significantly lower than that of Taxotere in P-69 cells. (FIG. 13C) SN12C and (FIG. 13D) HEK293 cells were plated in 96 well plates (3,000/well). After attachment overnight, the cells were incubated with IR-MUT1 for 48 hrs. The MTT assay was employed to determine the cytotoxicity of IR-MUT1 in SN12C and HEK293 cells grown in vitro. The results showed IR-MUT1 inhibited human renal cancer cell growth in culture with an identical IC50 of 12 nM as that of the taxotere. In contrast, cytotoxicity of IR-MUT1 on HEK293 cells is significantly higher (IC50 of 1,000 nM) than those of SN12C cells; taxotere inhibited the growth of HEK293 with an estimated IC50 of 600 nM.

FIG. 14 depicts an in vivo study showing SQ tumor reduction with IR-MUT1: Comparison with IR783 and taxotere treatment in accordance with an embodiment of the present invention. 1 million C4-2 human prostate cancer cells were implanted subcutaneously into the back of the 4 to 6 week old athymic nude mice. The inventors compared the effects of IR-MUT1 with the dye (IR783) or drug (Taxotere) alone on the growth of subcutaneous human prostate tumors in mice. 3 groups of male mice (5 mice per group) were injected i.p with 18783, IR-MUT1 and taxotere; IR783 and IR-MUT1 were injected at a dose of 5 mg/kg per mouse daily (or an accumulated dose of 30 mg/kg per week, calculated based on 6 days with one drug- or dye-free day) whereas taxotere was injected at a dose of 15 mg/kg twice per week (to avoid systemic toxicity) after tumor implantation. Mice treated with IR-MUT1 had substantially smaller tumors than those of the dye treated mice. Tumor diameters were measured with a caliper, and tumor volume in mm³ is calculated by the formula: Volume=(width)²×length/2. The incidence of tumor formation in IR-783 group (9/20) was also higher than the IR-MUT1 treated mice (2/20); no tumor formed in taxotere-treated group.

FIG. 15 depicts another in vivo study showing differential body weight reduction: Comparison between IR-MUT1, taxotere, and IR783 in accordance with an embodiment of the present invention. During treatment, the body weights were obtained daily. With the exception of mice assigned to the taxotere group which lost about 50% of the body weight, there was no body weight loss in mice treated with IR783 or IR-MUT1.

FIG. 16 depicts an in vivo study showing reduction of serum PSA in mice bearing human prostate C4-2 tumors treated with IR-MUT1 or taxotere in accordance with an embodiment of the present invention. Serum PSA levels were used to monitor tumor growth in a C4-2 SQ tumor model. Mice were checked for serum PSA levels before implantation and at 35 and 45 days after tumor cell implantation. In IR-MUT1 and taxotere group, the serum PSA levels of mice are significantly lower than in IR-783 (control) group.

FIG. 17A and FIG. 17B are photographs from an in vivo study showing IR-MUT1 caused apoptosis in SQ C4-2 tumors grown in mice. Note IR-MUT1 caused C4-2 tumor death as evidenced by the destruction of nuclear morphology FIG. 17A when compared to IR-783 control dye-treated specimen FIG. 17B in accordance with an embodiment of the present invention. The presence of apoptosis in SQ C4-2 tumor cells of IR-MUT1 group was confirmed by histopathology (H/E stain, 100×) (FIG. 17A). From the histomorphologic analysis, C4-2 tumor cells in IR783 group were not affected by this dye (FIG. 17B).

FIG. 18A is a graph and FIG. 18B is a series of images depicting another in vivo study showing intratibial tumor reduction by IR-MUT1 and taxotere injection in accordance with an embodiment of the present invention. 1 million C4-2 human prostate cancer cells were implanted intraosseously into the tibia of the 4 to 6 week old athymic nude mice. To assess inhibition of tumor growth by IR-MUT1, 3 groups of male mice (5 mice per group) were injected i.p. with IR783, IR-MUT1 or taxotere at the doses as described above from 30 days after tumor cell intratibial implantation. The animals were observed daily and body weights were measured daily. Tumor diameters are measured with calipers, and tumor volume in mm³ is calculated by the formula: Volume=(width)²×length×0.5236. FIG. 18A: In IR-783 injected group, there were 4 tumors growing from tibia (4/5) in comparison with only 1 (1/5) tumor growing in IR-MUT1 group. The average volume of tumor is significantly higher than in IR-MUT1 group. There were no tumors (0/5) formed in taxotere-treated group. FIG. 18B: From bone x-ray scans, the tumors appear to consist of mixed osteoblastic and osteolytic lesions. Notably, severe osteolytic lesions were apparent in IR783 treatment group compared with taxotere group and IR-MUT1 group. IR-MUT1 inhibited bone osteolytic lesions and attenuated osteoblastic lesions caused by the injected C4-2 tumor cells when compared to the IR783 treatment group.

FIG. 19 depicts marked OATPs (1B3, 2B1 and 5A1) expression differences between a human prostate cancer cell line, ARCaP-M, and a normal human prostate epithelial cell line, P-69, at the level of mRNA as determined by RT-PCT in accordance with an embodiment of the present invention. These differences are consistent with the dye, IR-783, and dye-drug conjugate, IR-MUT1, which potentially mediate the preferential uptake and accumulation via the presence of OATPs in tumor but not normal cells.

### DETAILED DESCRIPTION OF THE INVENTION

The following nonlimiting description provides additional details of some embodiments of the invention.

Small molecule cancer-targeting drugs have unique features compared to antibody, aptamer or peptide mediated cancer therapy as shown in Table 1.

TABLE 1
Comparison of Antibody, Aptamer and Small Molecule Mediated Cancer
Targeting and Drug Delivery

| feature and | | | | small |
|---|---|---|---|---|
| benefits | antibody | aptamer | peptide | molecule |
| size (g/mol) | >100,000 | >10,000 | 500-10,000 | <1,000 |
| time needed for | months | weeks | days to weeks | days |
| synthesis | | | | |
| Immunogenicity | yes | yes | possible | no |
| conjugation or | difficulty | difficulty | Less difficult | easy and |
| incorporation of | | | | controllable |
| therapeutic agent | | | | |
| cancer specificity | excellent | excellent | good | excellent |
| scalability | usually ug- | usually ug-mg | mg scale; | can be prepared in |
| | mg scale; | scale; poor | difficult to make | gram or kilograms |
| | poor | scalability | gram or | easily |
| | scalability | | kilograms scale | |
| handling and | difficult | difficult | less difficulty | |
| process | | | than antibody | easy |
| stability and | poor stability; | poor stability; | good stability; | stable in ambient |
| storage | low- | low- | require low | temperature |
| | temperature | temperature | temperature for | |
| | storage | storage | storage | |
| overall translational | poor | poor | good | excellent |
| capability for cancer | | | | |
| targeting and drug | | | | |
| delivery | | | | |

The benefits of using small molecules for cancer targeting and drug delivery is obvious (see Table 1). Compared to macromolecular targeting ligands, e.g., antibody and aptamer, small molecules are much easier to prepare and have no immunogenicity. The scalability, handling, sterilization and shelf-life stability all have significant effects on the clinical translation of therapeutic modalities. Small molecules are most promising for use in the clinical setting because of the simplicity of handling as well as their easy of scale-up, sterilization and storage.

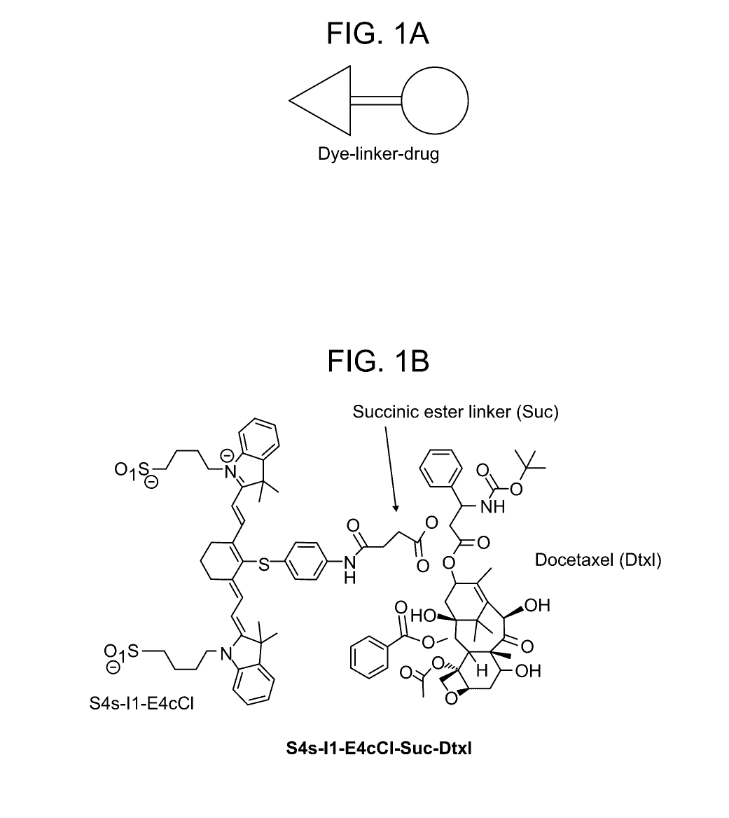

The present invention provides ligand-drug conjugates for targeted cancer therapy. The ligand targets cancer cells and allows for delivery of the drug to the desired location. The conjugates provided here have three components: a targeting ligand, a therapeutic agent (drug), and a linker that connects the ligand to the drug. FIG. 1A shows the general structure of the conjugates of the invention, and FIG. 1B shows one specific example. The word “ligand” and “dye” are used interchangeably throughout this specification.

The present invention also provides ligands for targeted cancer therapy. The ligands are as described herein for the ligand-drug conjugates.

The drugs which are used in the conjugates of the invention can be any therapeutic agent which can be linked to the targeting ligand. Examples of useful drugs include: FDA approved drugs for treatment of cancer; aminoglutethimide; asparaginase; bleomycin; busulfan; carboplatin; carmustine (BCNU); chlorambucil; cisplatin (cis-DDP); cyclophosphamide; cytarabine HCl; dacarbazine; dactinomycin; daunorubicin HCl; doxorubicin HCl; estramustine phosphate sodium; etoposide (VP-16); floxuridine; fluorouracil (5-FU); flutamide; hydroxyurea; hydroxycarbamide; ifosfamide; interferon a-2a, a-2b, leuprolide acetate (LHRH-releasing factor analogue); lomustine (CCNU); mechlorethamine HCl (nitrogen mustard); melphatan; mercaptopurine; mesna; methotrexate (MTX); mitomycin; mitotane (o.p′-DDD); mitoxantrone HCl; octreotide; plicamycin; procarbazine HCl; streptozocin; tamoxifen citrate; thioguanine; thiotepa; vinblastine sulfate; vincrinstine sulfate; amsacrine (m-AMSA); azacitidine; hexamethylmelamine (HMM); interleukin 2; mitoguazone (methyl-GAG, methyl glyoxal bis-guanylhydrazone (MGBG)); pentostatin; semustine (methyl-CCNU); teniposide (VM-26); paclitaxel, docetaxel, and other taxanes; vindesine sulfate and other small molecule drugs and biologics (for example antibodies, antisense nucleic acids and small interference or micro RNAs) that are designed to target cell growth and survival, angiogenesis, heat shock proteins, microtubules, cell adhesion, motility and migration, bisphosphonate drugs such as zolendrate and palmedranate for metastatic bone cancer treatment, peptide therapeutic agents such as cyclosporine and samatostatin, nucleic acids such as siRNA and oligonucleotide drugs.

The targeting ligand is linked to the drug through any suitable linker. In general, the linker has the following structure: x---y, where x and y can both react with groups on the ligand and drug to link the structures together. These groups on the ligand and drug include groups such as halogen atoms, COOH, NH2, OH and SH. Some examples of linkers include succinic ester, amino acid, peptide, diacid, bisamine, bis-alcohol, other anhydrides, CN or an alkyne group used for the click reaction, epoxy, hydrazine, azide, aldehyde, ketone, sulfonic acid, phosphoric acid, phosphoamidite, guanidine, short (C1-C6) alkyl, aromatic group, ester, amide, urea, thiourea, imidazole and its derivatives, thioester, acrylate, thiol ether, dithioate, selenide and phenyl selenide, diene, diketone, pyrimidine, purine and other hetrocycle ring structure, crown ether (for chelating with metal), phenoldiazene (photochromic probe), nitrobenzene and its derivatives (photo quencher or as photocaged probe), iodo or bromo (for radioactivity labeling and heavy atom phasing), monosaccharide and oligosaccharide (e.g., cyclodextrin), azirine and benzophenone (for photo crosslinking), bipyridine (metal chelating), biphenol and aminophenol (redox electron or radical electron traps), other indole derivatives (as electrochemical crosslinker) and any radioactive atom or chelator for those atoms (for MRI or PET imaging applications). It is known in the art how to prepare suitable linkers with suitable groups and react linkers with groups to be linked, as well as to functionalize both the linkers and groups to be linked to cause the desired linkage to occur.

The targeting ligand generally comprises a polyen (dien to tetraen, in one embodiment) that connects two aliphatic indoles on both ends of the polyen. In one embodiment, the targeting ligand is a cyanine dye or derivative thereof. In one embodiment, the cyanine dye derivative is IR783 or a derivative thereof. In one embodiment, the targeting ligand is an infrared or near-infrared absorbing dye. In one embodiment, the targeting ligand has a wavelength of maximum fluorescence emission greater than 650 nm. In one embodiment, the targeting ligand comprises two to four conjugated double bonds and two aliphatic indole structures. As used herein, a “derivative” means that one or more atoms or portions of the molecule are changed from the referenced structure.

The ligand-drug conjugates of the invention have therapeutic effects in the treatment of cancer. As used herein, “therapeutic effect” means reducing the signs, symptoms, or causes of a disease, or other desired alteration of a biological such as delay of disease progression by preventing or eliminating circulating cancer cells from the blood or facilitating the death of cancer cells in lymph node, bone marrow and/or soft tissues. As used herein, “cancer” means a disease characterized by abnormal growth of cells that is not regulated by the normal biochemical, physiological and physical influences from the host micro environment. Cancer which is capable of responding to treatment according to the compounds, compositions and methods disclosed herein include, for example, those listed in Isselbacher et al. (1994), Harrison Principles of Internal Medicine, 1814-1877. The compounds, compositions and methods disclosed herein are useful in the treatment of polycystic kidney disease and cancers such as, carcinomas, lymphomas, leukemias, neuroendocrine tumors, and sarcomas. A representative but non-limiting list of cancers is lymphoma, Hodgkin's Disease, myeloid leukemia, bladder cancer, brain cancer, head and neck cancer, kidney cancer, lung cancers such as small cell lung cancer and non-small cell lung cancer, myeloma, neuroblastoma/glioblastoma, ovarian cancer, thyroid and adrenal gland cancers, pancreatic cancer, prostate cancer, skin cancer, liver cancer, melanoma, colon cancer, cervical carcinoma, breast cancer, and other epithelial and mesenchymal cancers with unknown origin. Particularly, prostate cancer, pancreatic cancer and kidney cancer may be treated by the ligand-drug conjugates of the present invention. The compounds, compositions and methods disclosed herein may be used for the treatment of cancers through direct cytotoxic effects on localized and disseminated cancers but also can exert cytotoxicity to circulating cancer cells thus preventing the disseminated cancer cells from reaching metastatic sites. The compounds, compositions and methods disclosed herein may also be used for the treatment of inflammatory diseases such as osteoarthritis, rheumatoid arthritis, Crohn's Disease, pulmonary fibrosis, and Inflammatory Bowel Disease and benign/non-metastatic tumors such as benign prostate hyperplasia, and other benign tumors or precancerous conditions such as cervical and anal dysplasias, other dysplasias, severe dysplasias, hyperplasias, atypical hyperplasias, and neoplasias.

Also provided are methods of treatment, comprising: providing a small molecule conjugate compound of the invention and administering a therapeutic amount of the small molecule conjugate compound to a patient in need thereof. Also provided are compositions comprising a small molecule conjugate compound of the invention and a pharmaceutically acceptable salt or carrier. As used herein, a therapeutic amount means an amount which causes a therapeutic effect. Determination of therapeutic amounts is well known in the art. For example, the methods may be used to treat cancer. In particular, the methods of treatment may be used to treat prostate cancer, pancreatic cancer and renal cancer.

The ligand-drug conjugates of the invention have many uses in the treatment and diagnosis of cancer, which can be appreciated by a review of this disclosure. For example, the ligand-drug conjugates can be used to “sterilize” circulating tumor cells in patients to prevent or reduce the subsequent adhesion and extravasations of cancer cells to form metastatic deposits. The ligand-drug conjugates can be imaged directly in tumors. The intensity of the images correlates with drug concentrations in cancer tissues. This information provides physicians and therapists with a tool to adjust the dose of a drug, to follow-up and to predict clinical responsiveness of the target cancer cells in patients. Since the ligand-drug conjugates most likely enter the cancer cells by organic anion transporters, OATs and OATPs, this suggests differences may exist between normal and cancerous cells with respect to their OATs and OATPs profiles. Thus, ligand-drug conjugate accumulation in cancer cells reflects the heterogeneity of OAT and OATP which can predict the clinical behaviors of cancers. (See FIG. 19.)

As described elsewhere herein, cancer cells can be detected using the ligand-drug conjugate. In one embodiment, a patient's blood can be collected and analyzed after therapy to determine: a. If there are circulating cancer cells in patient's blood; b. If the cells are accumulating the ligand-drug conjugate in abundance, or c. If the cells are dying after administration of the ligand-drug conjugate. This information may be used for individualized therapy for diagnosis, prognosis and patient follow-up.

The IR783 dye is stable even after fixing in formalin. Therefore, a combined histopathology is presented which integrates the responsiveness of cancer cells to the ligand-drug conjugate (e.g., cell death assay) and the histopathology of the tissue sections (e.g., status of differentiation or malignancy such as Gleason score of human prostate cancer) and the relationship of these parameters can be defined with the concentration of the ligand-drug conjugates present or accumulated in tissues and cells at the site of action.

NIR dye-drug conjugates having fluorescence emission with λₘₐₓ at >700 nm do not experience significant interference from the autofluorescence of biologic materials. Thus, the concentration of the ligand-drug conjugates of the invention can be conveniently determined in tissues or cells without prior purification of the ligand-drug conjugates provided that insignificant amount of the compound of interest was metabolized.

Prolonged trapping of ligand-drug conjugates of the invention in cells or tissues represents a fundamental interaction between ligand-drug conjugates and the cell chemical constituents, which provides valuable prognostic and diagnostic information.

In further embodiments, the ligand-drug conjugates of the invention may be used in conjunction with other cancer therapeutics modalities, such as hormone deprivation, hormonal antagonists, radiation and chemotherapy. For example, the ligand-drug conjugates of the invention may be administered to a patient in need thereof, prior to, in conjunction with, or subsequent to another cancer therapeutic modality.

As used herein, “pharmaceutically acceptable salts” are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic pharmaceutically acceptable salts may also be formed, including hydrochloride, sulfate, nitrate, bicarbonate, and carbonate salts. Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Ligand Structure

The structure of the ligand can be changed to provide fine-tuning of the characteristics of the ligand-drug conjugate. For example, electron withdrawing groups or electron donating groups can be added to the ligand.

Scheme 1 shows several dye examples with excellent targeting and poor targeting.

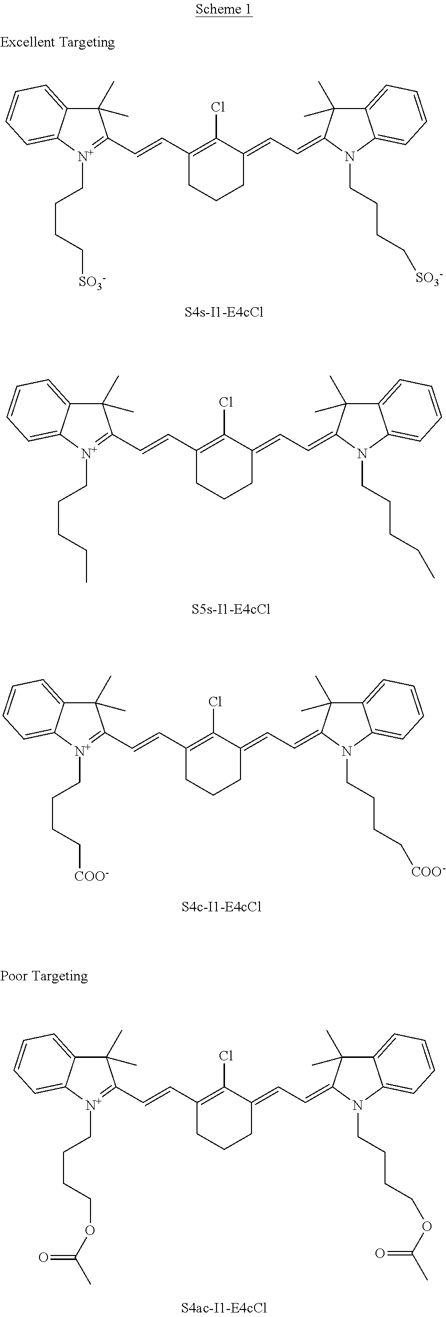

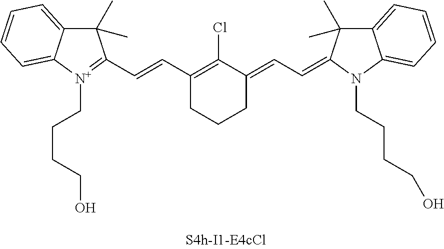 [SMILES: CC1(C)C(/C=C/C2=C(Cl)C(=C/C=C3/N(CCCCO)c4ccccc4C3(C)C)/CCC2)=[N+](CCCCO)c2ccccc21]

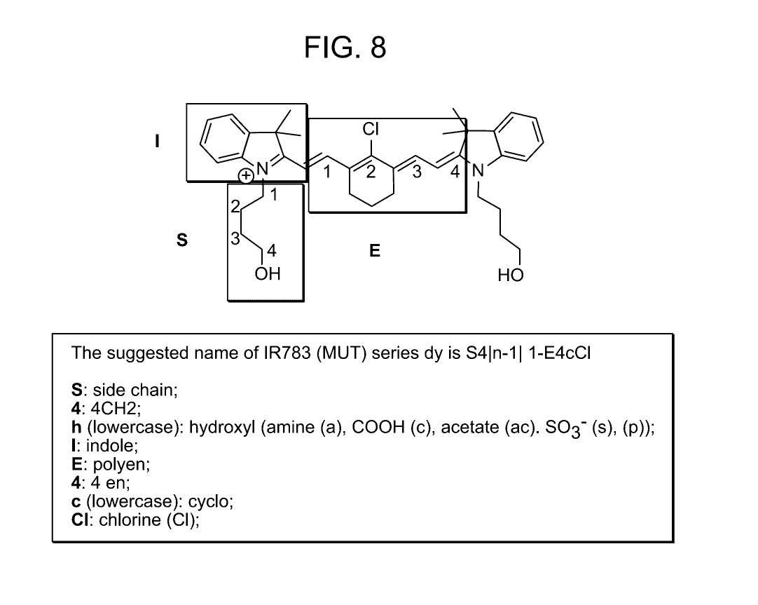

In one embodiment, the targeting ligand (dye molecule) comprises an indole portion (I), a polyen portion (E), and a side chain portion (S) (see e.g., FIG. 8).

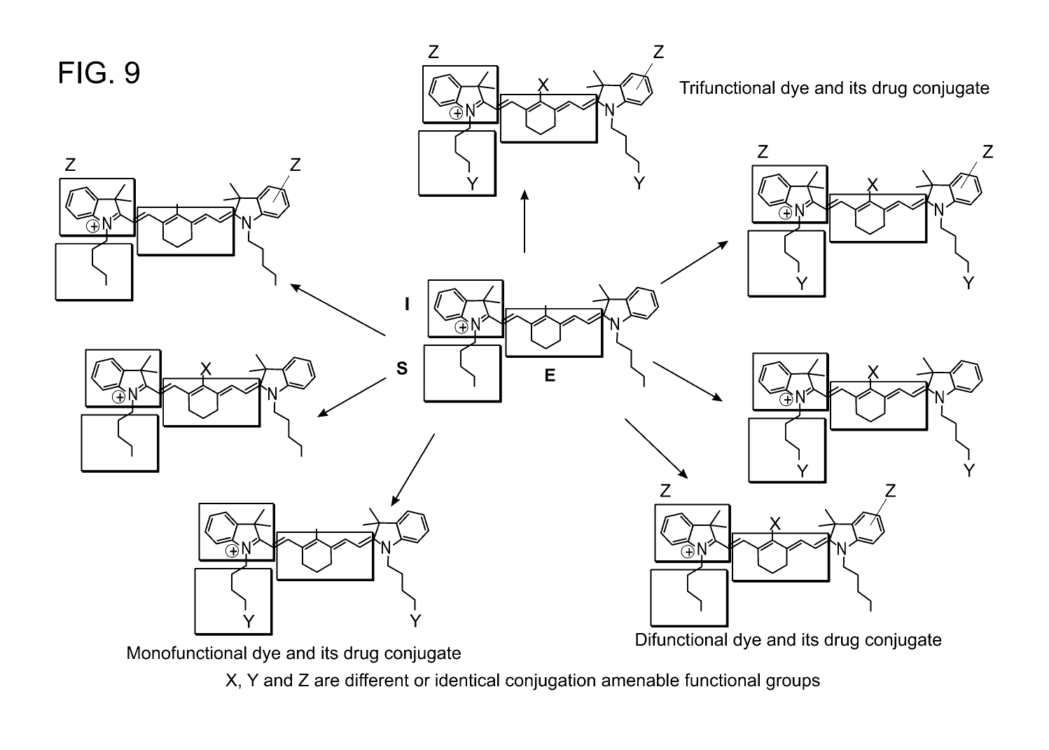

The composition and structure of drug-dye conjugates can be controlled by using dye analogues with conjugation amenable functional groups controlled at the specific positions (see e.g., FIG. 9). For example, conjugation amenable groups (—OH, —NH₂, —SH, —COOH) can be easily introduced to the I, E and S portions.

Accordingly, in certain embodiments, the indole portion, polyen portion and/or side chain portion comprise a conjugation amenable functional group; for example, —OH, —NH₂, —SH, —COOH.

In certain embodiments, the indole portion and the polyen portion are represented by the following formula:

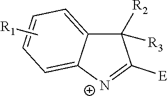

wherein E represents the polyen portion and R₁, R₂, and R₃ are each independently selected from the group consisting of: OH; NH₂; SH; COOH; H; C1-C15 alkyl and is optionally substituted with one or more nitrogen-containing groups, oxygen-containing groups, sulfur-containing or halogen atoms; alkoxy and is optionally substituted with one or more nitrogen-containing groups, oxygen-containing groups, sulfur-containing or halogen atoms; aryl and is optionally substituted by one or more heteroatoms or substituents; aromatic ring and is optionally substituted by one or more heteroatoms or substituents; non-aromatic ring and is optionally substituted by one or more heteroatoms or substituents; oxy; carbonyl; alkenyl; nitro; and amino.

In various embodiments, the polyen portion is a polyen substituted with a substituent selected from the group consisting of OH, NH₂, SH, and COOH.

In other embodiments, the polyen portion is a dien, trien, or tetraen optionally substituted with a substituent; optionally contains an aryl that is optionally substituted by one or more heteroatoms or substituents; optionally contains an aromatic ring that is optionally substituted by one or more heteroatoms or substituents; or optionally contains a non-aromatic ring that is optionally substituted by one or more heteroatoms or substituents; wherein the substituent is selected from the group consisting of OH, NH₂, SH, and COOH.

In various embodiments, the side chain portion and the indole portion is represented by the following formula:

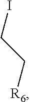

wherein I represents the indole portion and R₆ is selected from the group consisting of: OH; NH₂; SH; COOH; H; C1-C15 alkyl and is optionally substituted with one or more nitrogen-containing groups, oxygen-containing groups, sulfur-containing or halogen atoms; alkoxy and is optionally substituted with one or more nitrogen-containing groups, oxygen-containing groups, sulfur-containing or halogen atoms; aryl and is optionally substituted by one or more heteroatoms or substituents; aromatic ring and is optionally substituted by one or more heteroatoms or substituents; non-aromatic ring and is optionally substituted by one or more heteroatoms or substituents; oxy; carbonyl; alkenyl; nitro; and amino.

As also depicted in FIG. 8, in accordance with the naming scheme of the dye molecule, the name of IR783 (MUT) series dye is S4h-I1-E4cCI.

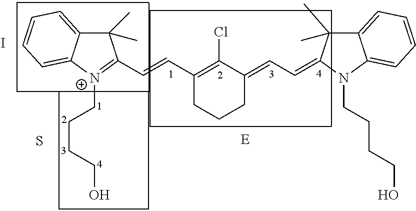

Shown below are exemplary structures for the indole (I) portion of the molecule:

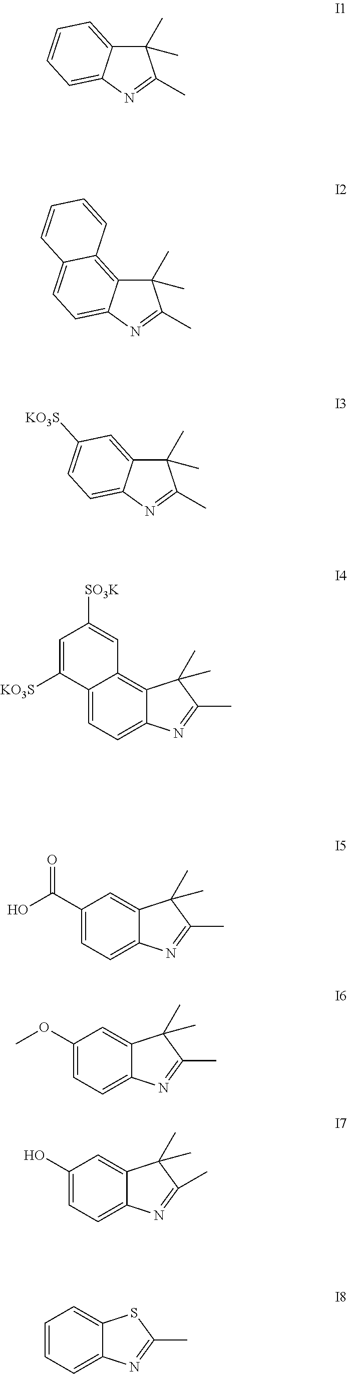

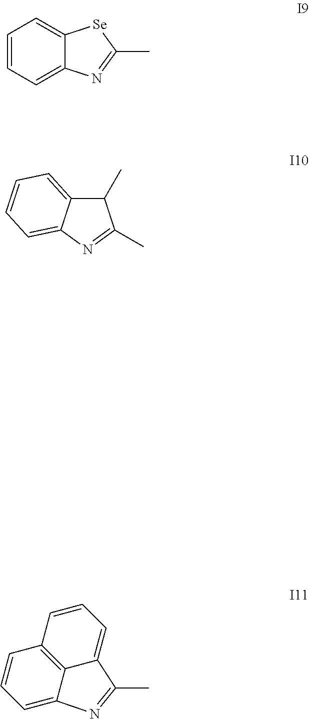

Shown below are exemplary structures for the polyen (E) portion of the molecule, where “I” indicates an indole portion.

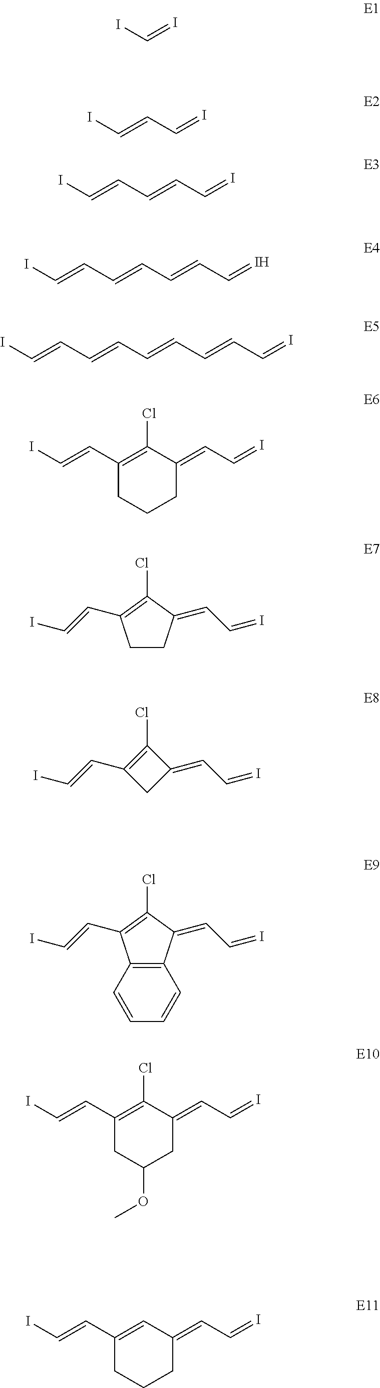

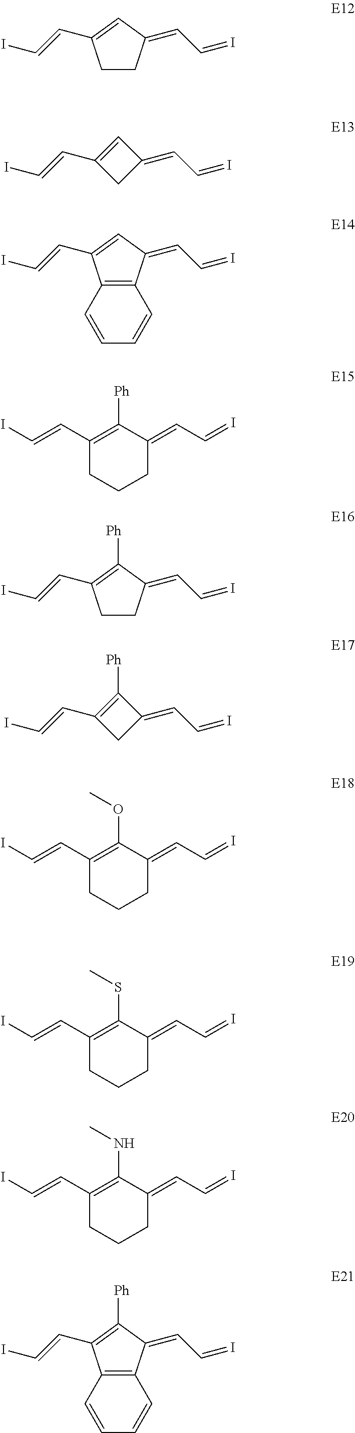

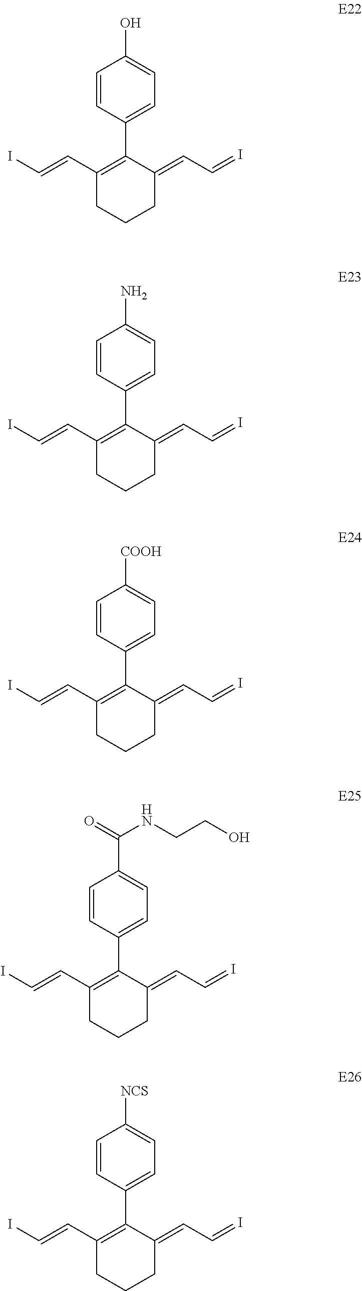

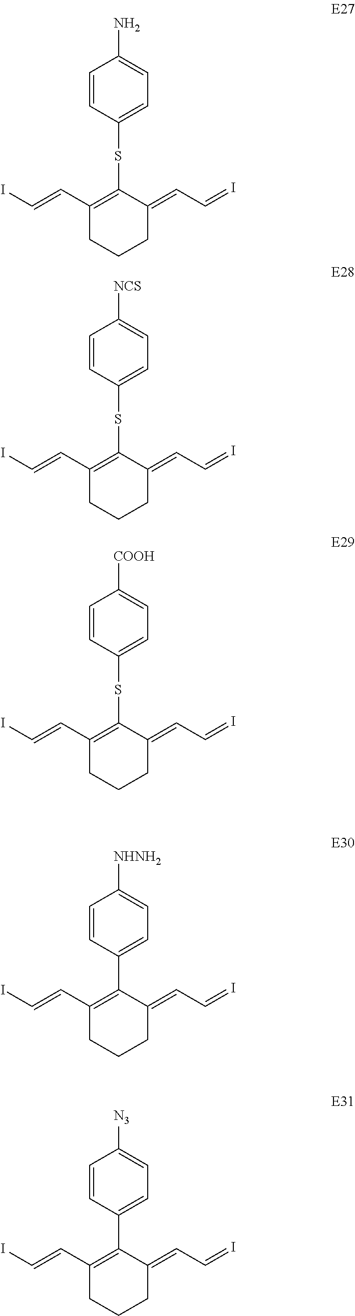

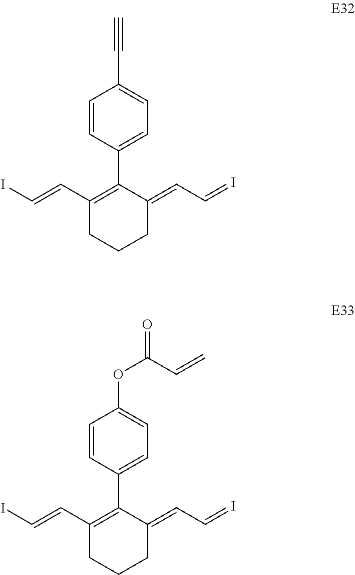

Shown below are exemplary structures for the side chain (S) portion of the molecule, wherein the “I” represents the indole portion:

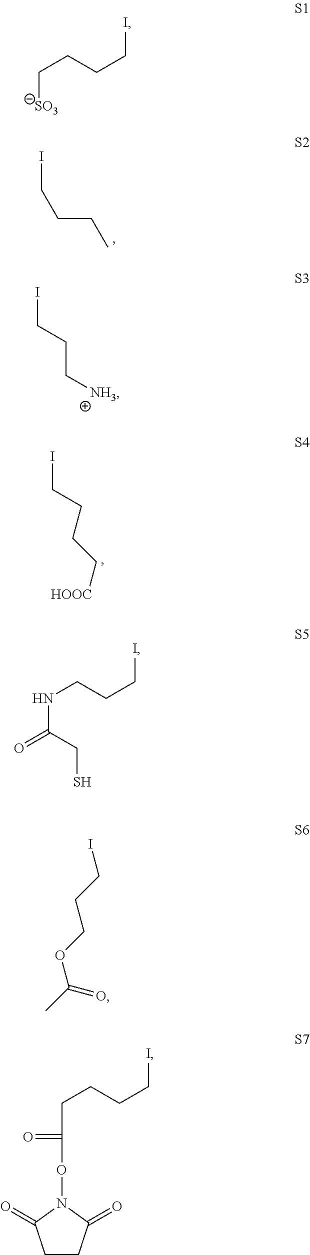

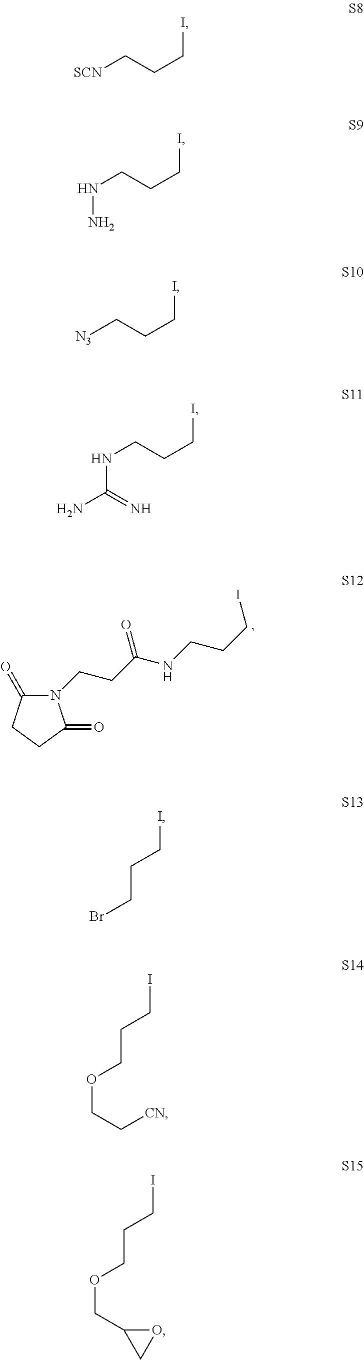

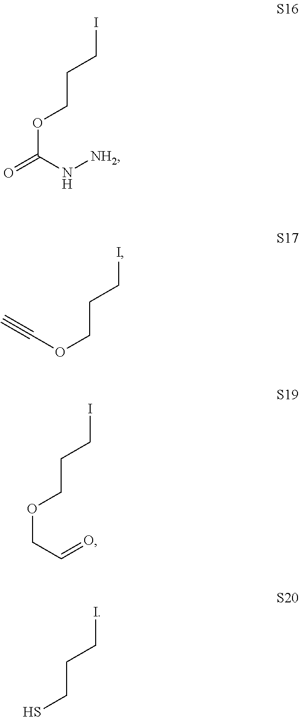

All combinations and subcombinations of the various portions of the dye molecule are intended to be included to the same extent as if they were drawn as separate compounds. To illustrate, any example of the polyen (E) can be combined with one or more examples of the indole (I) structure and one or more optional side chain (S) structures to form a dye molecule useful in the invention. In one embodiment, one E structure is combined with two examples of the indole structure and two examples of the side chain structure. In one embodiment, the two side chain structures are the same. In one embodiment, the two side chain structures are different. In one embodiment, the two indole structures are the same. In one embodiment, the two indole structures are different. In one embodiment, two different indole structures are attached to a polyen structure, and a different side chain structure is attached to each indole structure. In one embodiment, two of the same indole structures are attached to a polyen structure, and two of the same side chain structures are attached to each indole structure.

Synthesis

Synthesis of Ligand

Cyanine dyes can be synthesized following the general reaction scheme illustrated in Scheme 3.

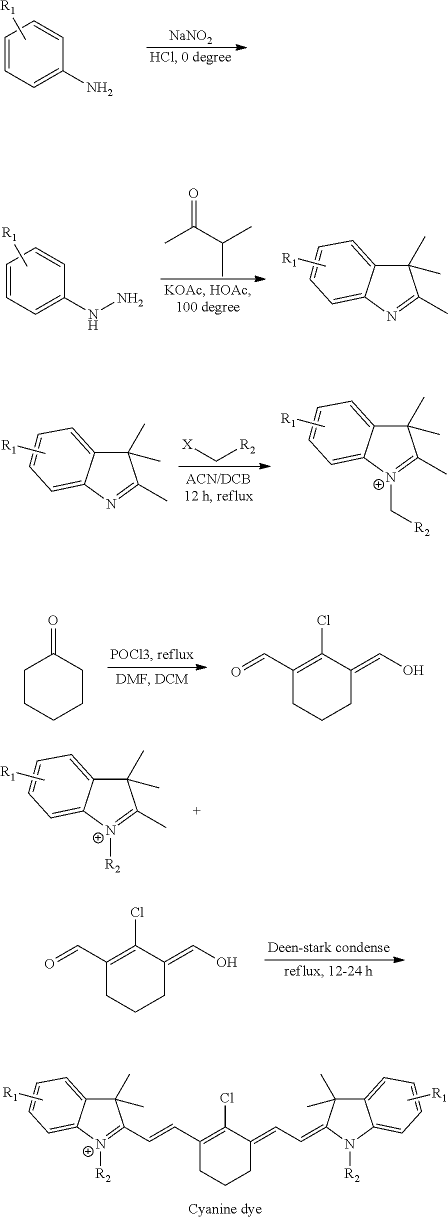

R₁ and R₂ are each independently selected from the group consisting of H; C1-C15 alkyl or alkoxy which may be substituted with one or more nitrogen-containing groups, oxygen-containing groups, sulfur-containing or halogen atoms (such as SO₃, OC(═O), NCS, NH₂, COOH); aryl or other ring systems such as six- or five-membered aromatic or non-aromatic rings which may be substituted by one or more heteroatoms or substituents described herein; oxy; carbonyl; alkenyl; nitro; amino; and other groups, such as those described and shown herein, wherein each of the groups may be optionally substituted by one or more halogen atoms or heteroatoms.

Shown below are examples of synthesis and different substituent groups that can each be separately combined with other groups to form other molecules of the invention.

### Example 1

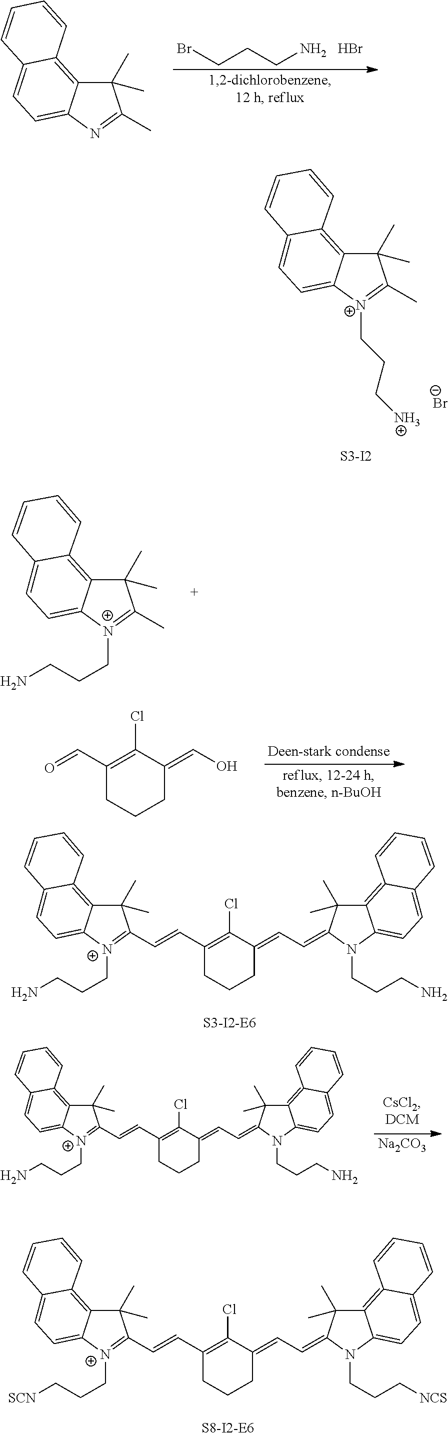

### Example 2

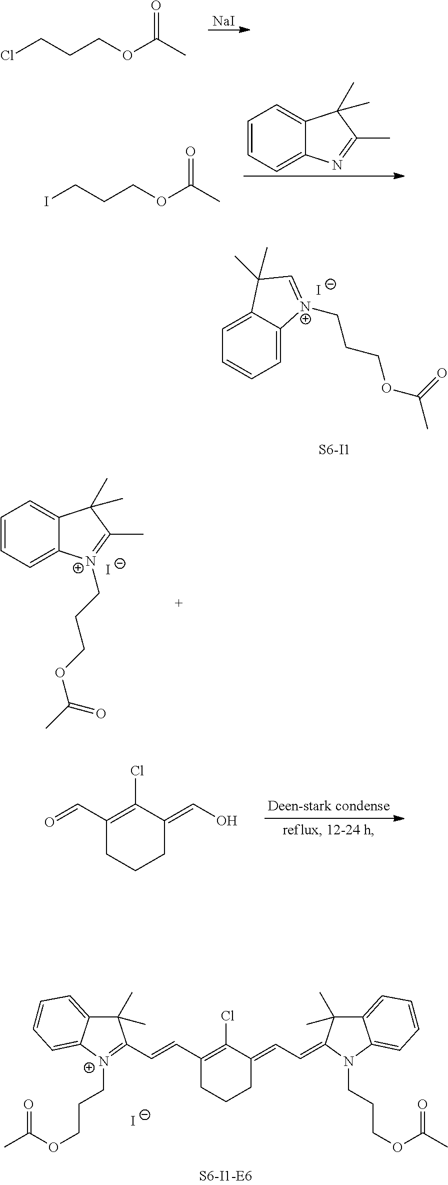

### Example 3

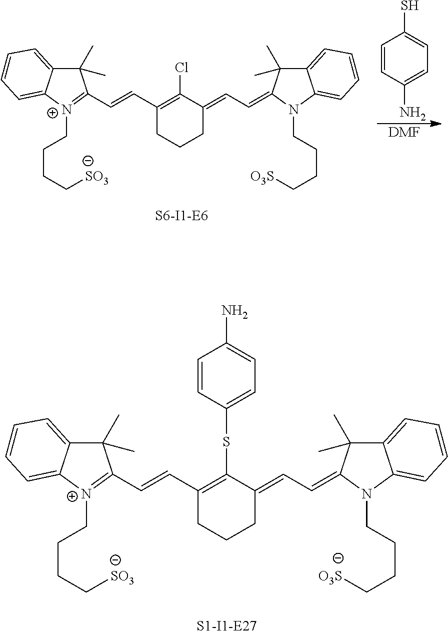

### Example 4

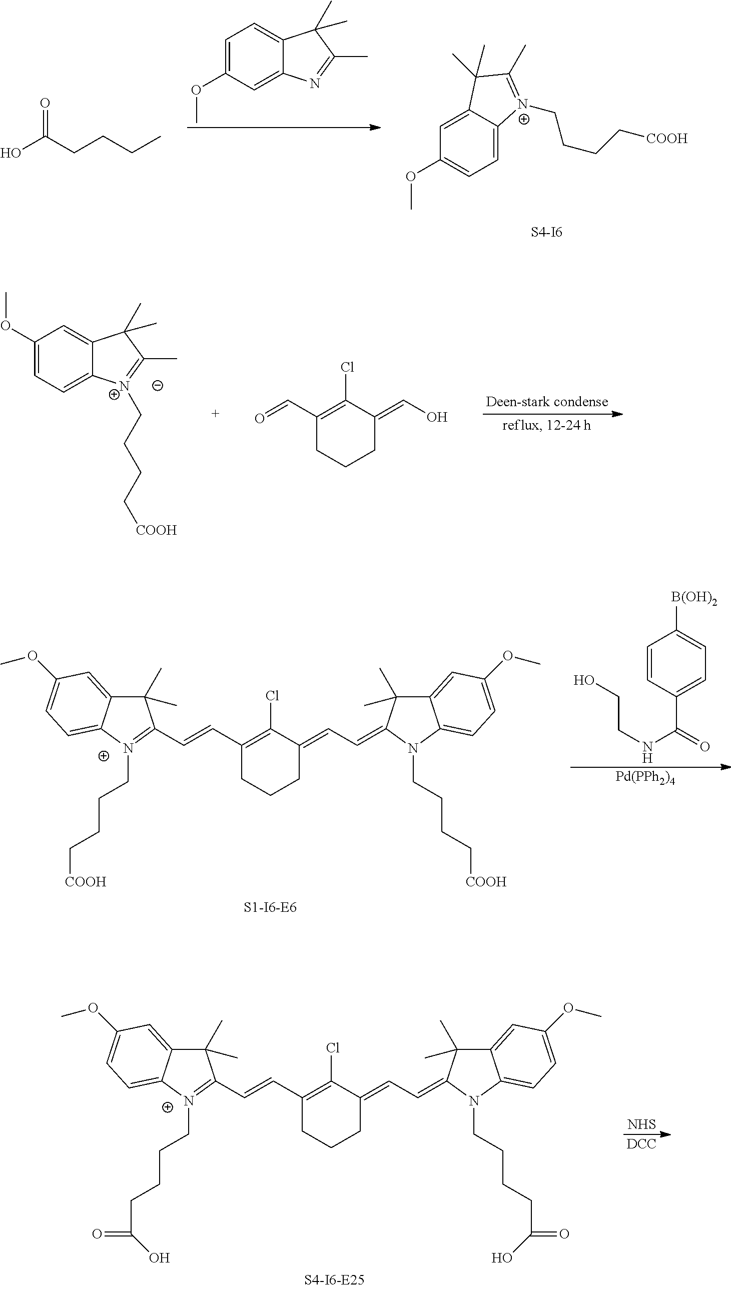

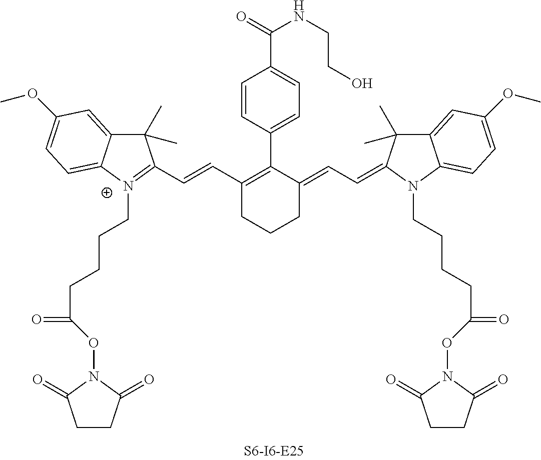

As shown in Scheme 3, a library of dyes can be easily prepared by changing R₁ and R₂ groups. In addition, the length and structure of polyen as well as the substituent on polyen can also be changed to optimize ligand cancer targeting.

Synthesis of Ligand-Drug Conjugates

Scheme 4 shows the general steps in a synthesis method for a conjugate of the invention.

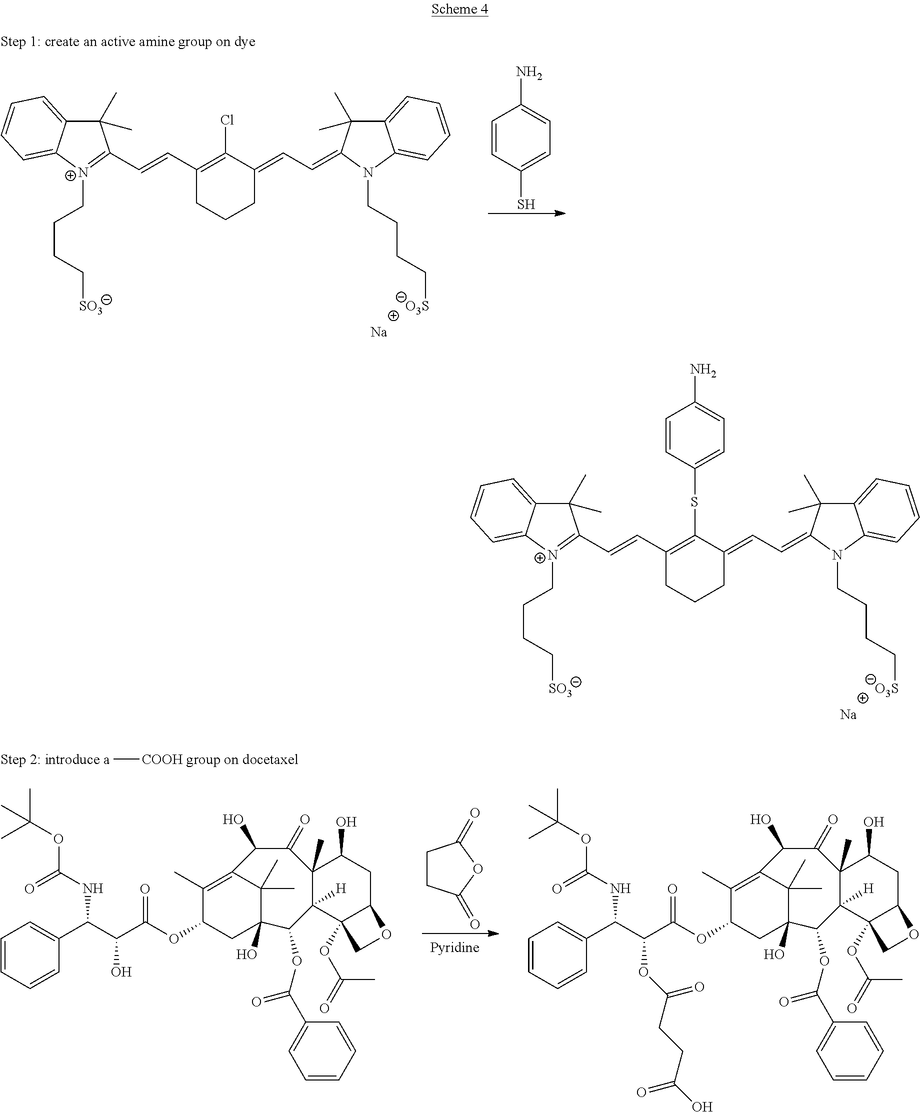

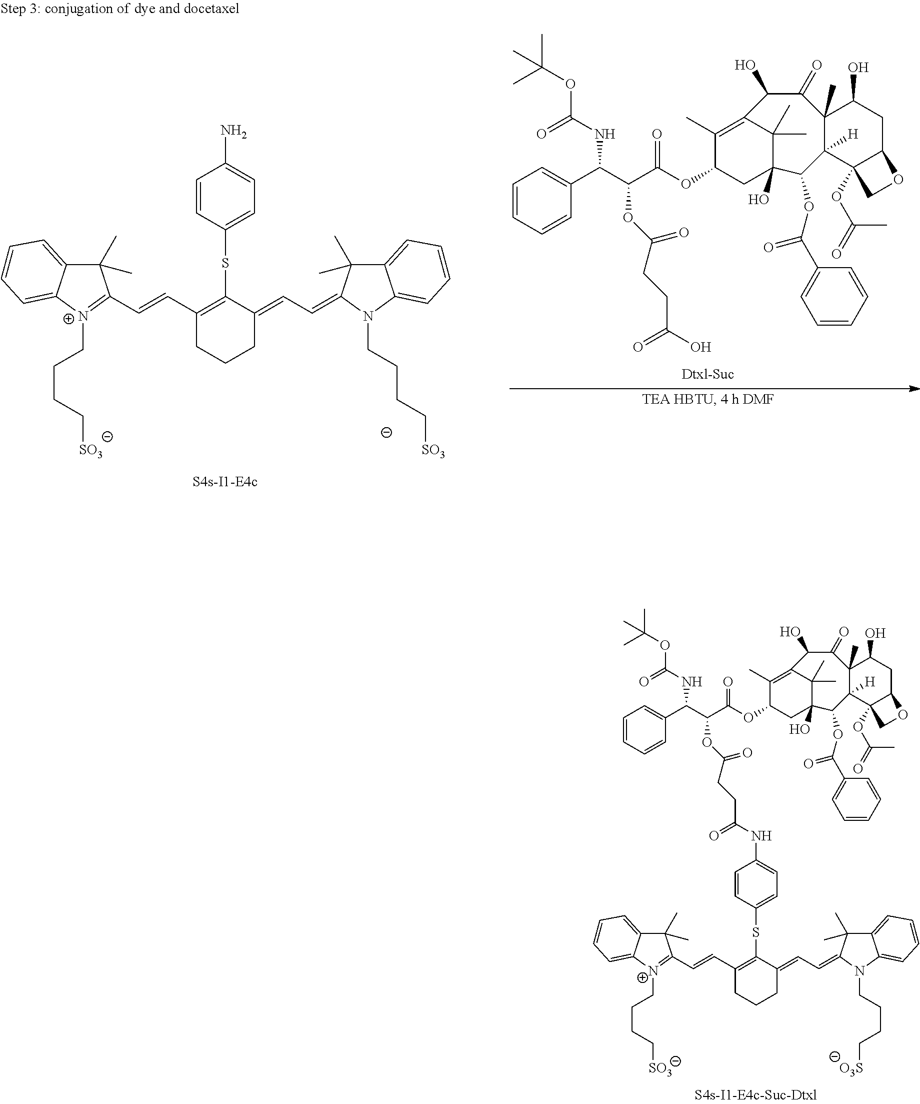

In a particular example, the —Cl of S4s-I1-E4cCI (Scheme 3) was converted to a more reactive amine functional group for the conjugation of therapeutic agents as exemplified by docetaxel (Scheme 5). The —Cl group of S4s-I1-E4cCI was converted to an aromatic amine group. Docetaxel (Dtxl) was then reacted with a succinic anhydride (Suc) to form a COOH-terminated Dtxl. The modified S4s-I1-E4cCI and Suc-Dtxl were conjugated using conventional coupling chemistry (Scheme 5). As shown in Scheme 5, a library of dyes can be easily prepared by changing R1 and R2 groups. In addition, the length and structure of polyen as well as the substituent on polyen can also be changed to optimize ligand cancer targeting.

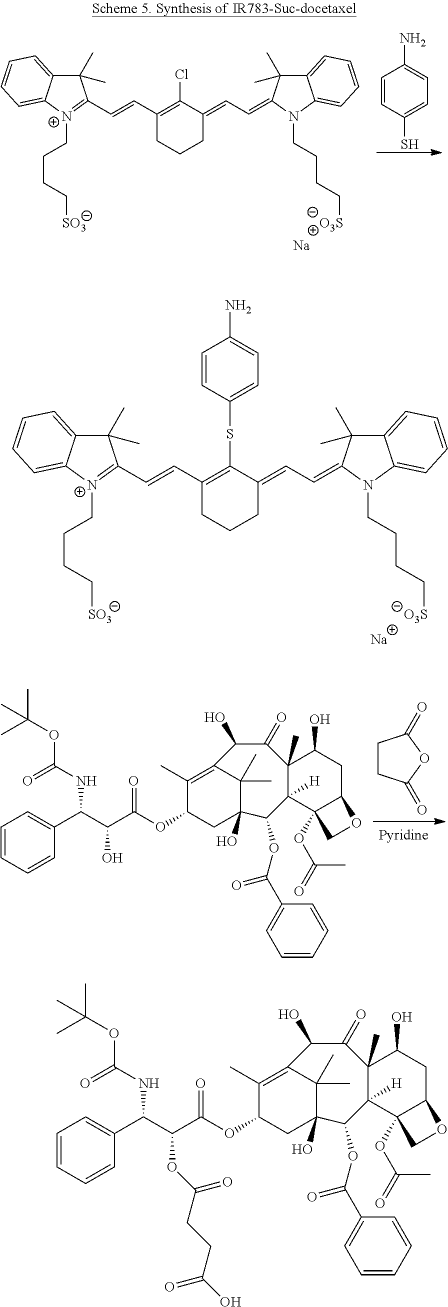

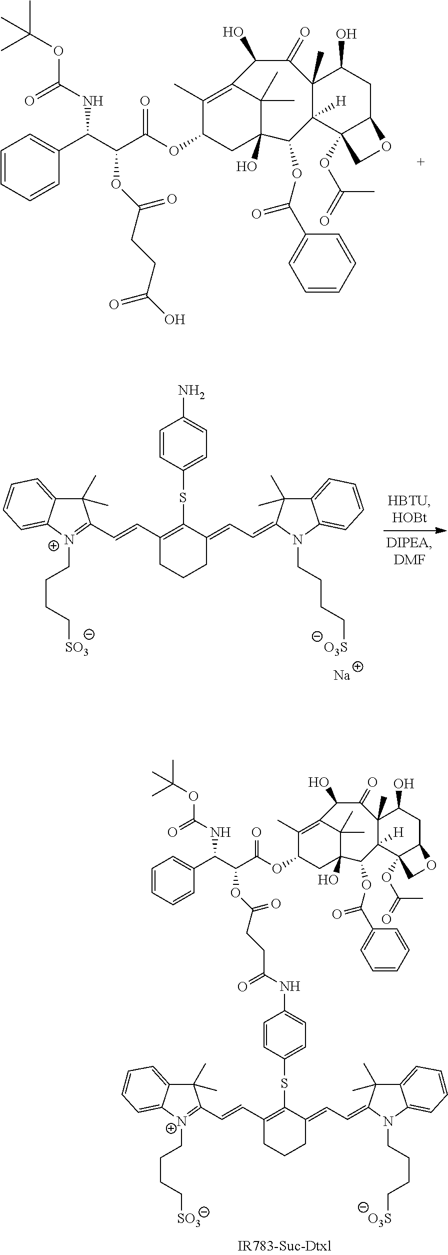

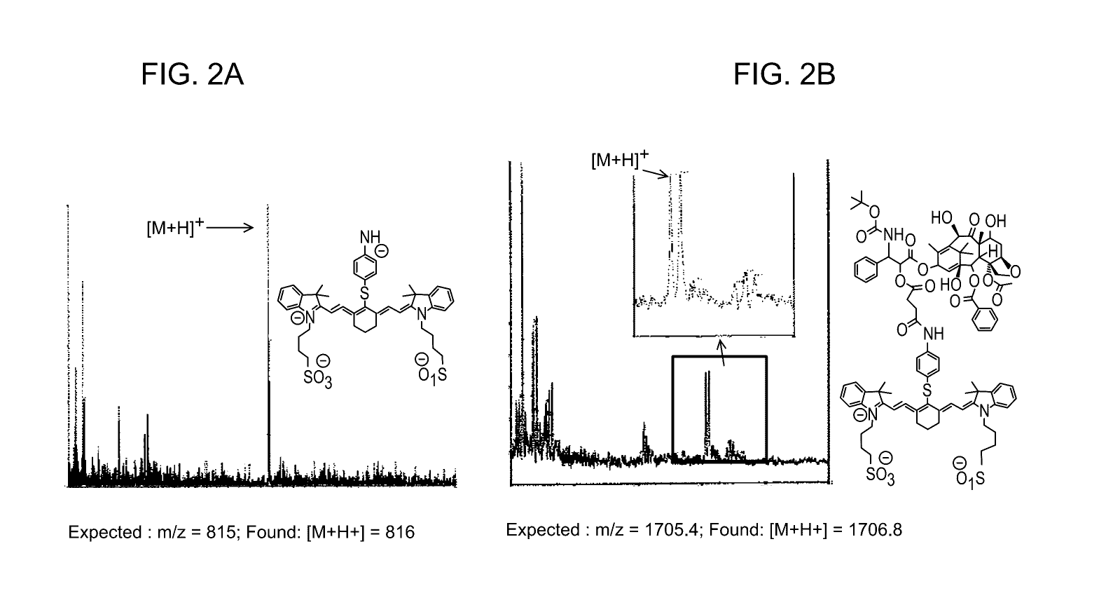

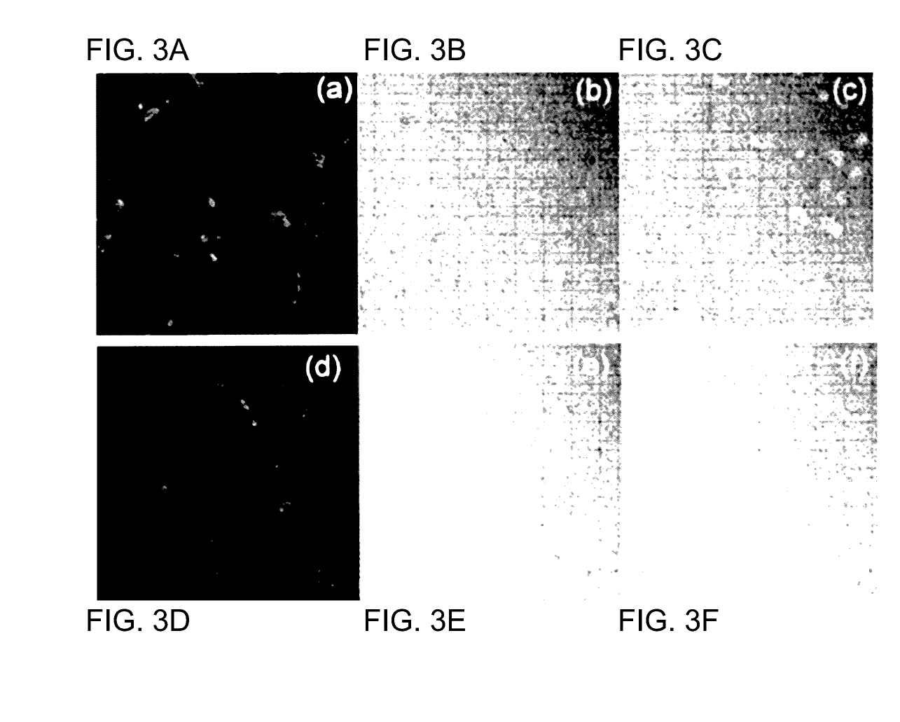

The structures of S4s-I1-E4cCI-Suc and S4s-I1-E4cCI-Suc-Dtxl were confirmed using mass spectrometry (FIG. 2A and FIG. 2B).

It is understood that the desired drug can be linked to the desired ligand and used in the methods of the invention. For example, IR-783 can be conjugated to each desired drug. IR-783 has been conjugated to docetaxel (IR-MUT1) and paclitaxel (IR-MUT2). These conjugates inhibit human prostate and bladder cancer cell growth in culture (data not shown).

In Vitro and In Vivo Evaluation of S4s-I1-E4cCI-Suc-Dtxl (IR-MUT1)

The targeting efficiency of S4s-I1-E4cCI-Suc-Dtxl was evaluated in vitro and in tumor-bearing mice.

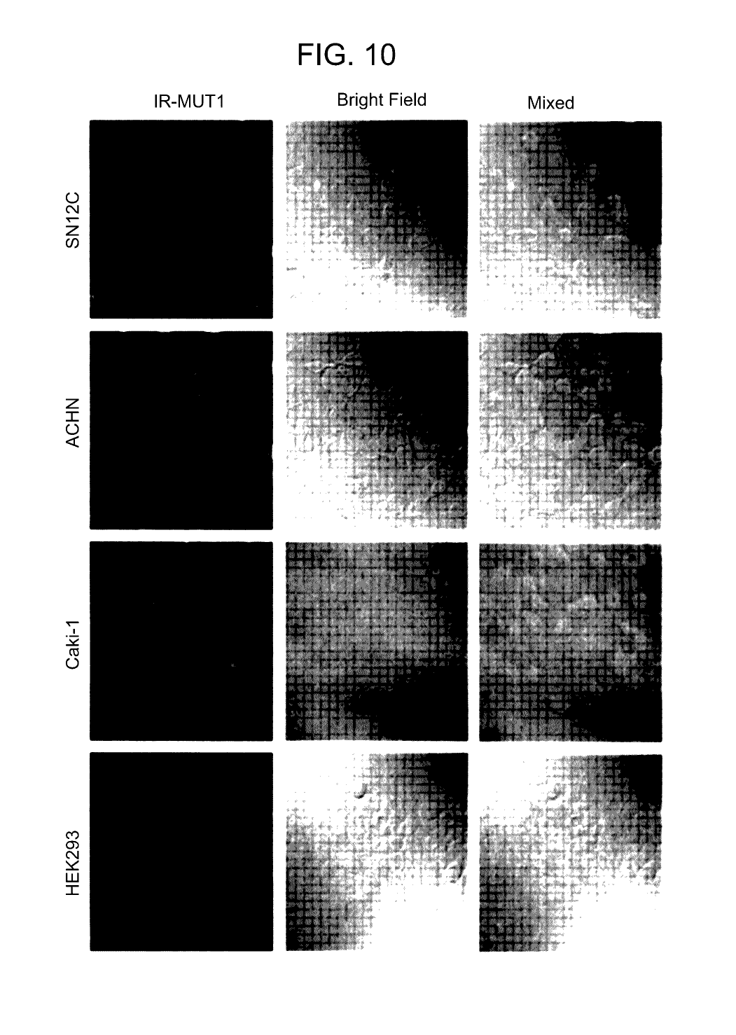

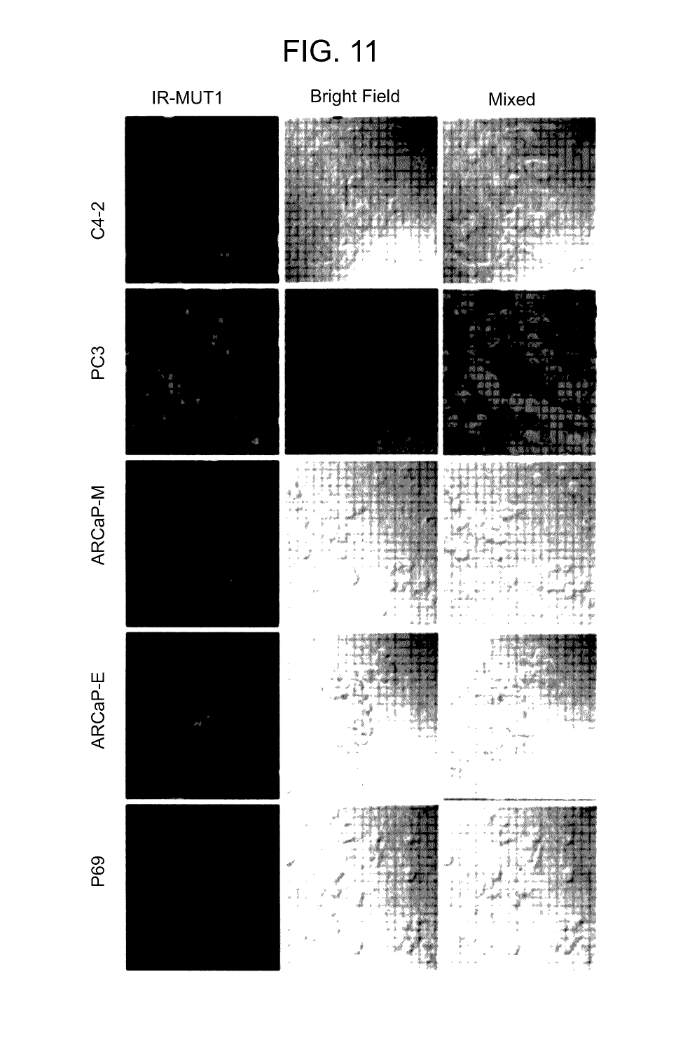

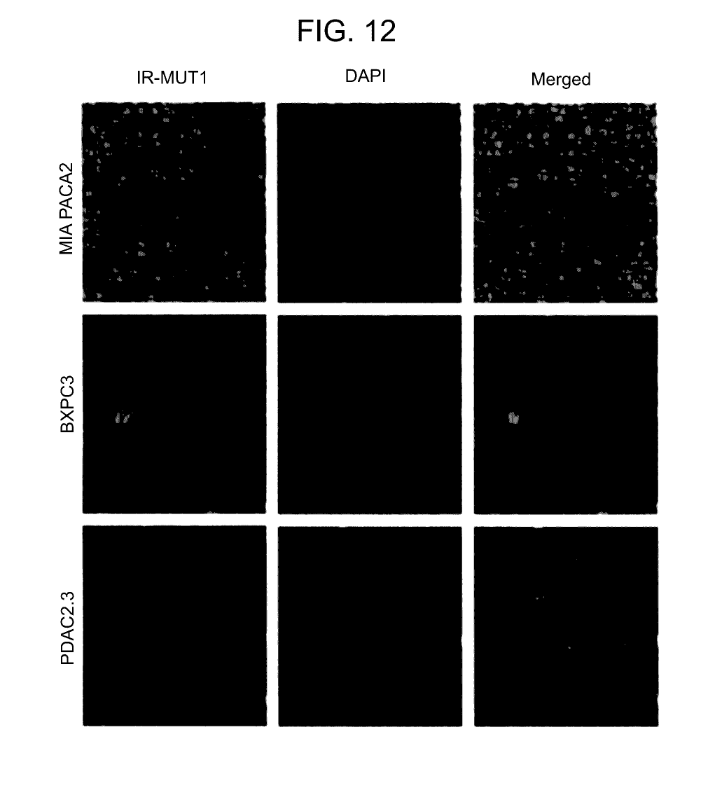

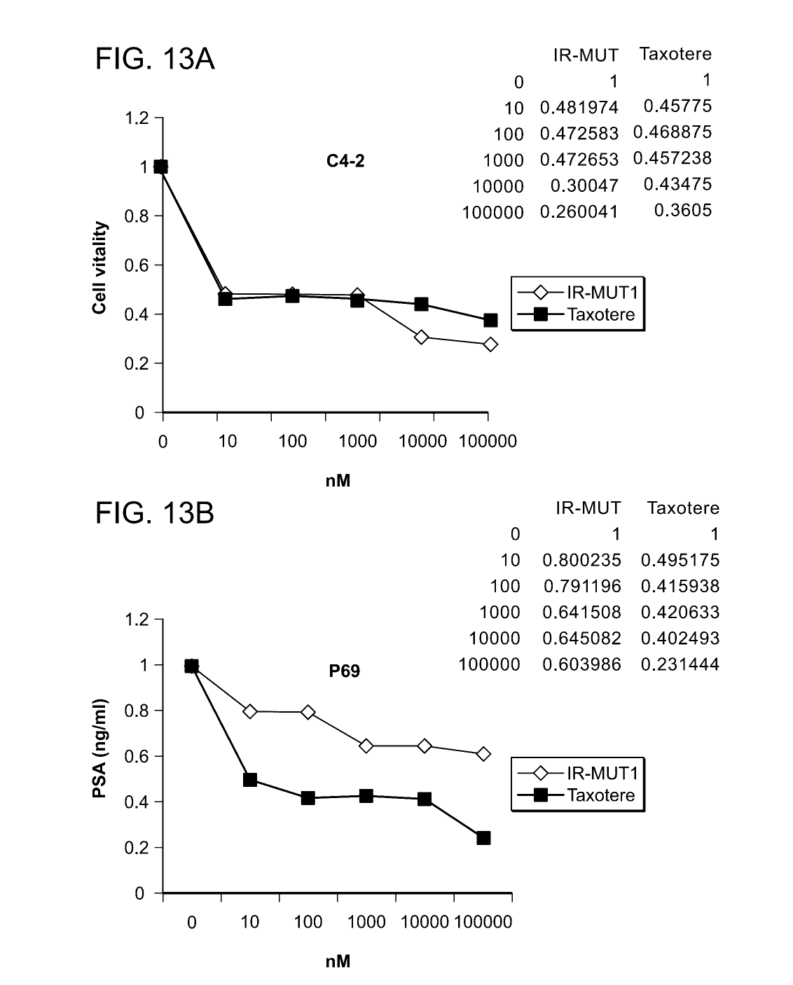

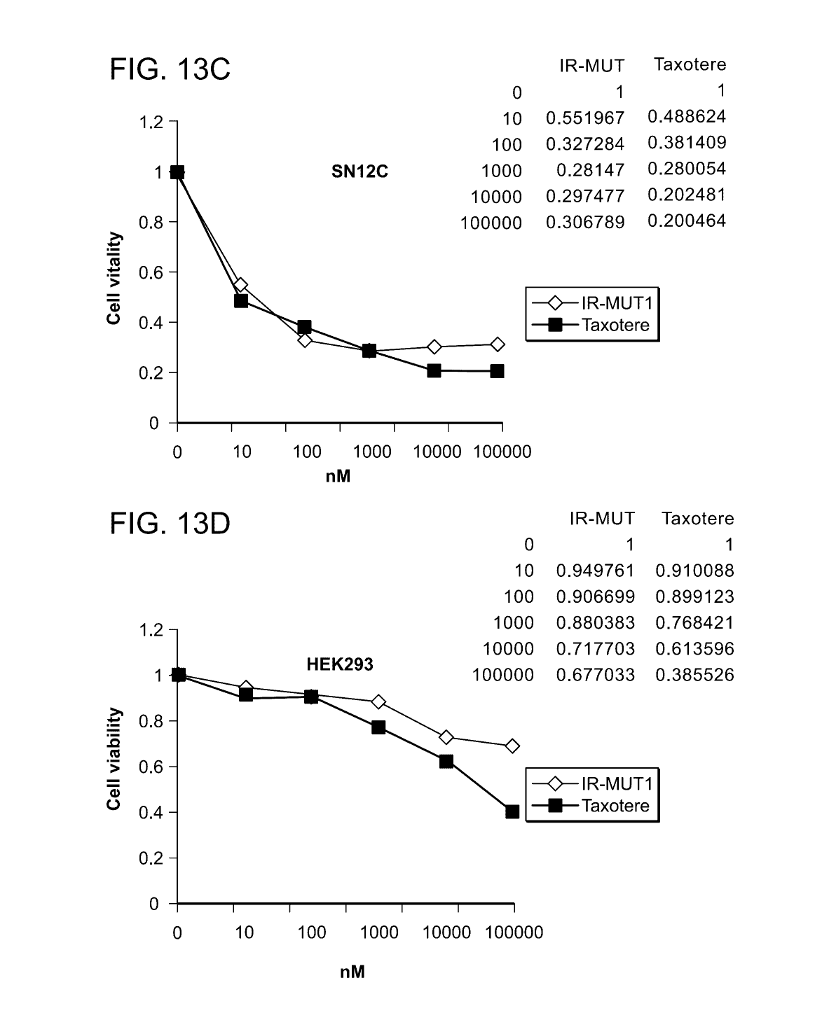

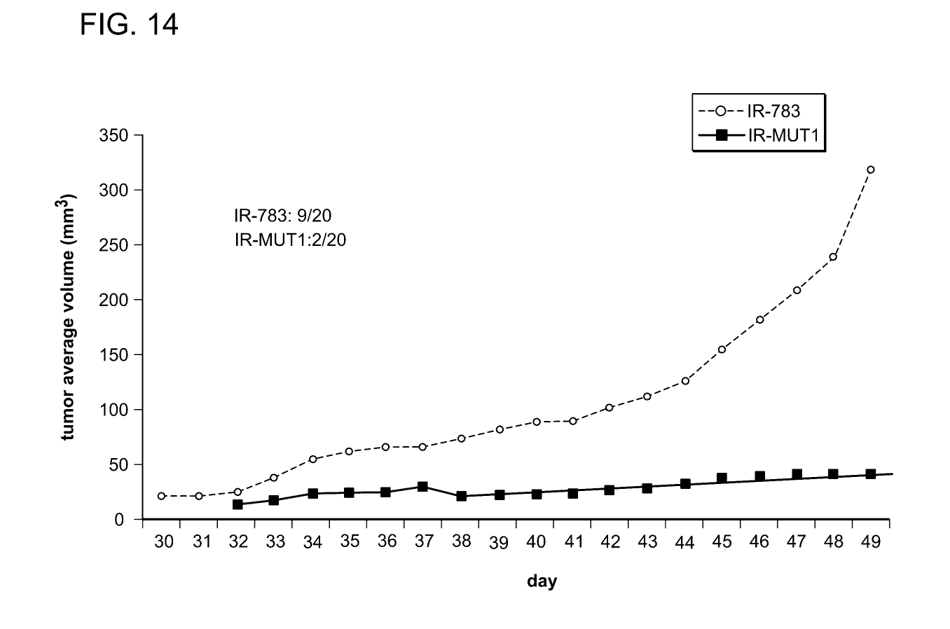

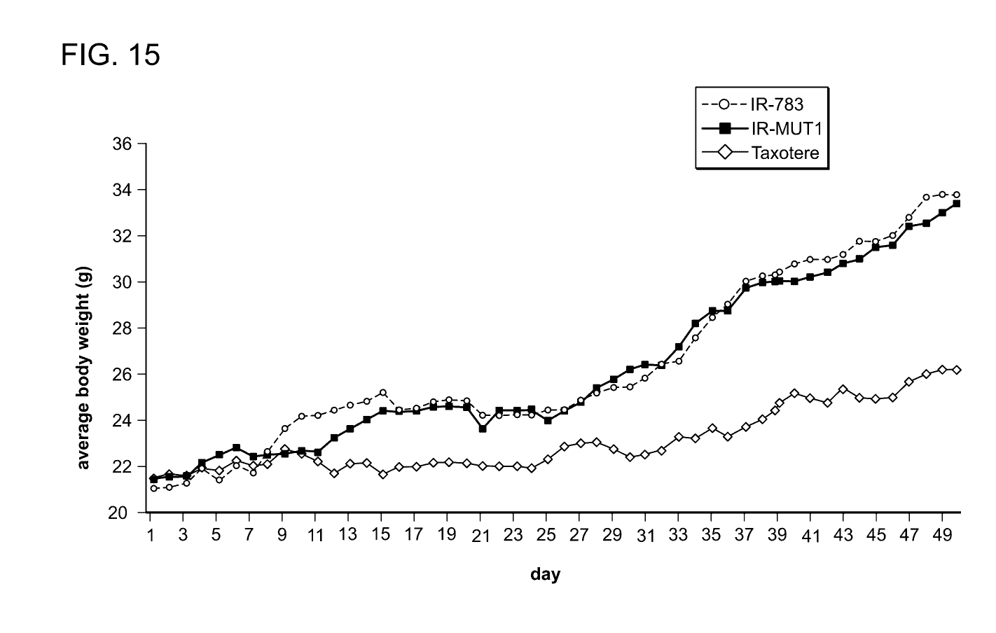

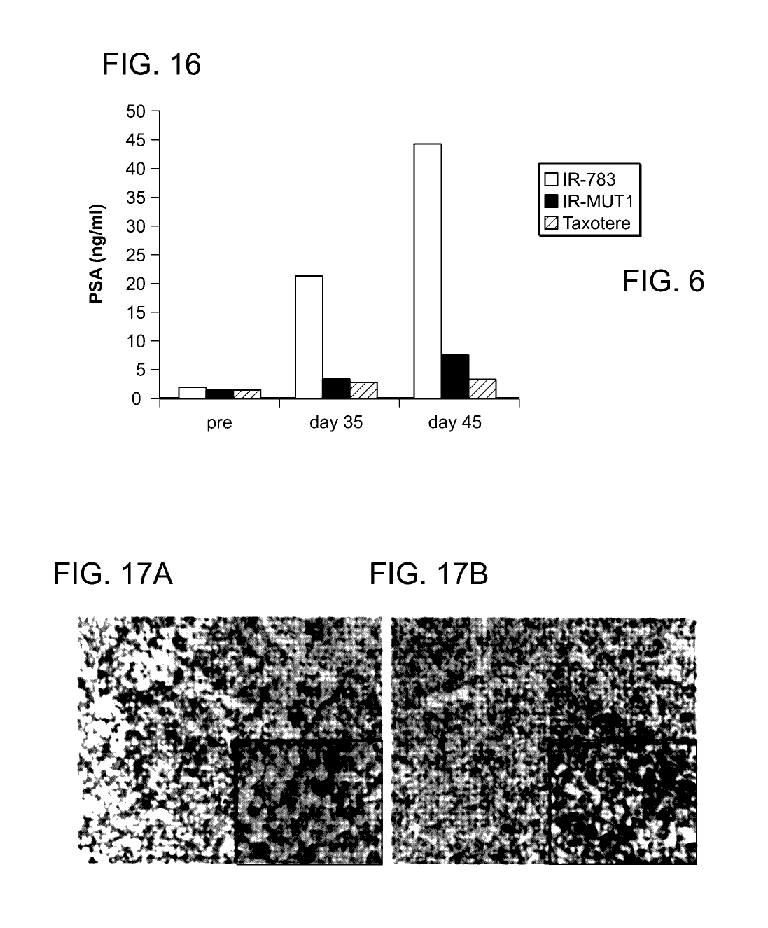

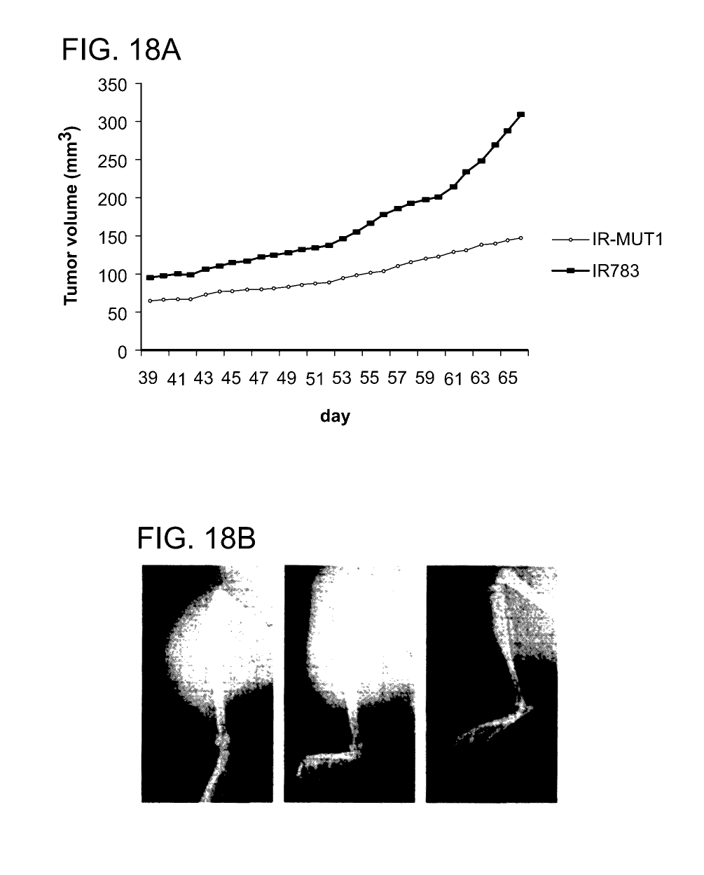

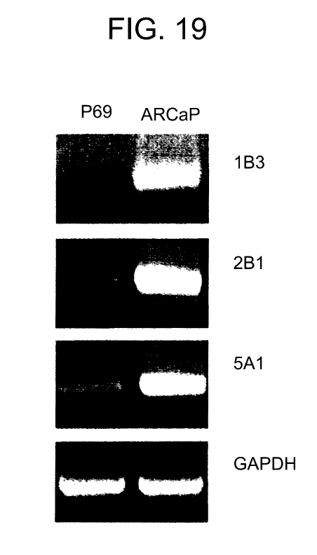

The internalization of IR-MUT1 (S4s-I1-E4cCI-Suc-Dtxl) was further evaluated in different cancer cells. Prostate cancer cells (C4-2, PC3, ARCaP-M, ARCaP-E), renal cancer cells (SN120, ACHN, Caki-1) and pancreatic cancer cells (MIA PACA2, BXPC3, PDAC2.3) were subject to IR-MUT1(20 μM) for 30 minutes, respectively (see FIGS. 10-12). Significant internalization of IR-MUT1 into all above cells was observed using a confocal fluorescence microscope. In contrast, the uptake of IR-MUT1 by normal human prostate epithelial cells (P69, see FIGS. 10-12), human embryonic kidney cells (HEK-293, see FIG. 10) was marginal to undetectable.

The in vitro cytotoxicities of IR-MUT1 were also measured in different cell lines. For SN120 (human renal cancer cell line) and C4-2 (human prostate cancer cell lines), the 1050 values in 48 hours of IR-MUT1 were 12 nM and 10 nM respectively. Noticeably, the 1050 values of IR-MUT1 were similar to taxotere (docetaxel) confirming the effectiveness of IR-MUT1 in targeting cancer cells. In a parallel study, however, HEK293 (a human embryonic kidney cell line) and P69 (a normal human prostate epithelial cell line), the IC50 values in 48 hours of IR-MUT1 were accordingly over 1000 nM and 100 nM; whereas the 1050 values of taxotere for those two cells were approximately 600 nM and 10 nM (see FIGS. 13A-D).

The in vivo targeting of S4s-I1-E4cCI-Suc-Dtxl was evaluated (FIGS. 14-18). S4s-I1-E4cCI-Suc-Dtxl showed highly effective targeting efficiency of human prostate tumors grown subcutaneously in mice (FIGS. 14-16 and 17A-17B) and human prostate tumors grown intratibially (FIG. 18). In both cases, not only the size of tumors, but also the % of incidence of tumor formation was significantly decreased (see FIG. 14 and FIG. 18). In comparison to the unconjugated taxotere-treated group. IR-MUT-1 is safe and did not affect the body weight of treated mice whereas taxotere, even treated with only half of the dose and reduced schedule of IR-MUT-1, reduced nearly 50% of the body weight, see FIG. 15). At the histomorphologic level, the inventors observed that IR-MUT1 killed prostate tumor cells by removing nuclear debris from tumor cells (FIGS. 17A-17B). Cytotoxic effects of IR-MUT1 in prostate tumor growth in mice was further substantiated by the serum PSA data where the inventors observed that both taxotere and IR-MUT1 treated mice had greatly depressed serum PSA (which has been shown by many previous studies to correlate with the size of prostate tumors) when compared to the dye only-treated mice (FIG. 16).

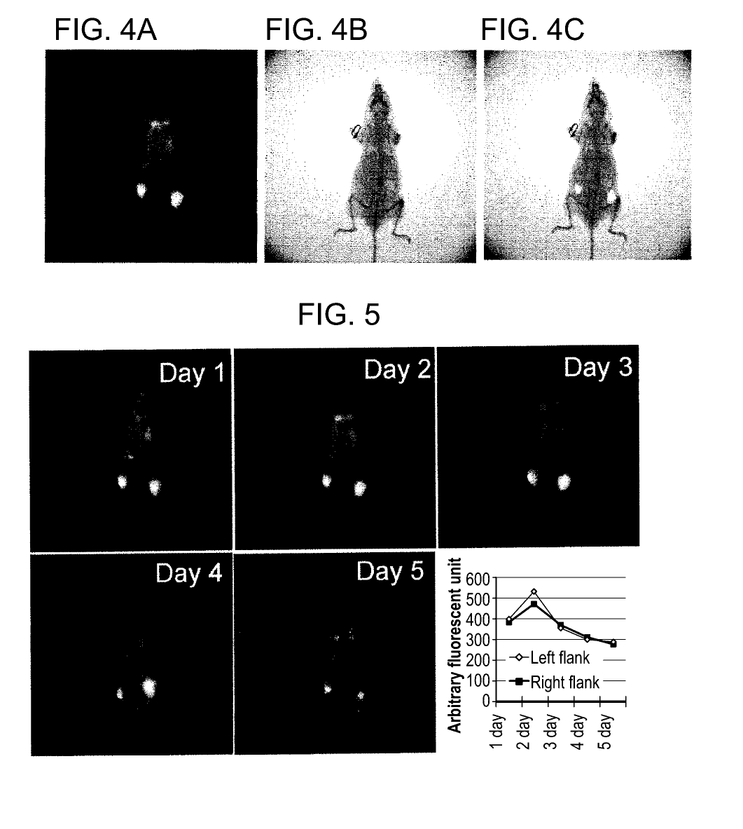

The whole body imaging of the treated animal demonstrated that S4s-I1-E4cCI-Suc-Dtxl was preferentially localized in tumor tissue. Unlike antibody or aptamer mediated cancer targeting in which substantial amount of administered materials are trapped in liver or spleen, S4s-I1-E4cCI-Suc-Dtxl retention in liver and spleen were low as compared to tumor tissue (FIGS. 4A-4C and FIG. 5). Furthermore, S4s-I1-E4cCI-Suc-Dtxl showed surprisingly long retention in tumor tissue. Even on Day 5 after injection, the fluorescence intensity (the amount S4s-I1-E4cCI-Suc-Dtxl) in tumor tissue decreased by only 25% as compared to the fluorescence intensity of the same tumor tissue on Day 1.

The in vivo efficacy of IR-MUT1 was evaluated in prostate C4-2 tumor model. C4-2 prostate cancer cells were subcutaneously implanted into the back of the 4 to 6 week old athymic nude mice. To assess the tumor reduction efficacy of IR-MUT1, male mice were divided into 3 groups (5 mice per group), and injected (i.p.) with (1) IR-783 (2) IR-MUT1 and (3) taxotere, with a dose of 5 mg/kg daily (one day off every 7 days) for IR-783 and IR-MUT1 but because of systemic toxicity, taxotere exposure was reduced to two injections per week at a dose of 15 mg/kg. The Inventors observed the incidence of tumor in IR-783 treated group was 9/2, whereas for IR-MUT1-treated group was significantly reduced to 2/20, and the volumes of tumors were also significantly reduced in IR-MUT1-treated group. Although there was no tumor growing in taxotere group, the body weights of mice in that group were noticeably lower than those in IR-MUT1 and IR-MUT groups (FIG. 14 and FIG. 15). The serum prostate specific antigen (PSA) levels, which indicating the presence of prostate cancers, were monitored during the tumor reduction study. For the IR-MUT1 and taxotere groups, the serum PSA levels at 35 and 45 days were dramatically lower than those in IR783 group, and attained to the PSA levels before tumor implantation. The results confirmed the reduction of prostate tumors by IR-MUT1 or taxotere treatments (FIG. 16). Immuno-histopathology analysis of tumors tissues from the IR-MUT1 group showed the apoptosis of C4-2 prostate cancer cells, which was negligible in tissues from the IR-783 groups (FIGS. 17A-B).

In another tumor model, C4-2 cancer cells were administered intraosseously into the tibia of the 4 to 6 week old athymic nude mice. Mice were divided into 3 groups (5 mice per group) and were injected (i.p) with IR-783. IR-MUT1 and taxotere at a dose described above (see [00102]) from 30 days after tumor cell implantation. For the IR-783 group, there were 4 tumors growing from tibia (4/5), in comparison with only 1 tumor growing in the IR-MUT1 group (1/5). The average volumes of tumor were significantly higher in IR-783 treated mice than those in the IR-MUT1 treated group. From the X-ray imaging study of the tibia bone area, both the osteolytic and osteoblastic lesions were apparently observed in the IR-783 treatment group (FIG. 18B); while no lesion were observed for the IR-MUT1 and taxotere groups. It indicates that IR-MUT1 can potentially inhibit the bone osteolysis and osteoblastogenesis caused by the presence of tumor cells in mouse skeleton.

FIG. 5 shows a time course study of in vivo cancer targeting and retention of S4s-I1-E4cCI-Suc-Dtxl. Whole-body NIR optical imaging and X-ray of athymic nude mice with subcutaneously implanted human bladder cancer T24 cells 48 h after intravenous injection of S4s-I1-E4cCI-Suc-Dtxl at Day 1-5. Experimental condition: 1×10⁶ human bladder T24 cells were subcutaneously injected into athymic nude mice at both flanks of the animal. After tumor sizes reach approximately 7-8 mm³ in diameter, mice were injected intravenously into tail vein with S4s-I1-E4cCI-Suc-Dtxl at a dose of 10 nmol per mouse. Whole-body NIR optical imaging and X-ray of the animals was conducted on a Kodak In Vivo Animal Imaging Station (New Haven, Conn.) equipped with 800 nm filter sets (excitation/emission, 800/850 nm). Images were analyzed using Kodak ID3.6.3 network version imaging at Day 1, 2, 3, 4 and 5. The fluorescent intensity of S4s-I1-E4cCI-Suc-Dtxl in tumors in both left and right flank was measured on each day.

Synthesis and Evaluation of Structure-Function Correlation of Cyanine Dyes

After confirming cancer targeting in vivo using IR-MUT1 (S4s-I1-E4cCI-Suc-Dtxl), the structure-function correlation of IR783 was evaluated by changing the indole ring, aliphatic side-chain, and polyen structure (Table 2).

TABLE 2
IR783 derivatives for in vitro and in vivo cancer targeting

| | In vitro | In vivo |
|---|---|---|
| IR783 derivatives | targeting | targeting |
| 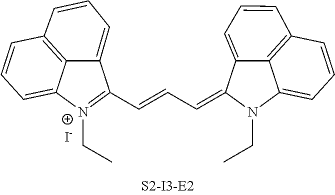 [SMILES: CCn1/c(=C/C=C/C2=[N+](CC)c3cccc4cccc2c34)c2cccc3cccc1c32 ; [I-]] | good | poor |
| 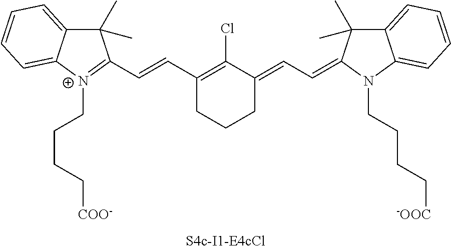 | excellent | excellent |
| 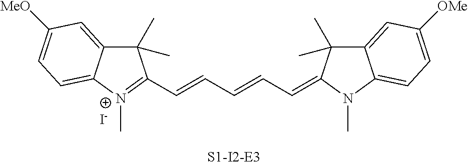 [SMILES: COc1ccc2c(c1)C(C)(C)C(/C=C/C=C/C=C1/N(C)c3ccc(OC)cc3C1(C)C)=[N+]2C ; [I-]] | excellent | to be tested |
| 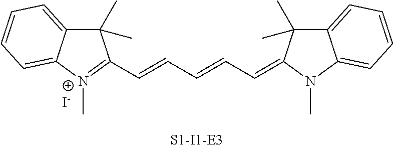 [SMILES: CN1/C(=C/C=C/C=C/C2=[N+](C)c3ccccc3C2(C)C)C(C)(C)c2ccccc21 ; [I-]] | poor | poor |
| 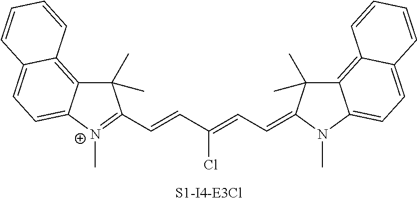 [SMILES: CN1/C(=C/C=C(Cl)/C=C/C2=[N+](C)c3ccc4ccccc4c3C2(C)C)C(C)(C)c2c1ccc1ccccc21] | excellent | to be tested |
| 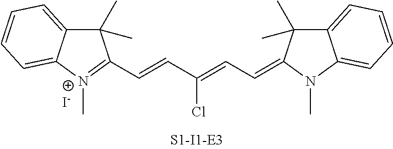 [SMILES: CN1/C(=C/C=C(Cl)/C=C/C2=[N+](C)c3ccccc3C2(C)C)C(C)(C)c2ccccc21 ; [I-]] | excellent | to be tested |
| 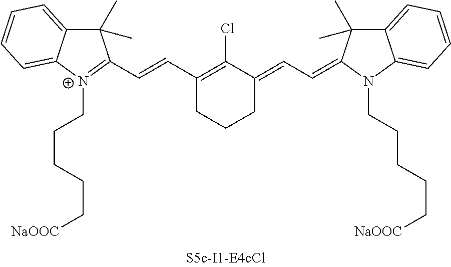 | excellent | excellent |
| 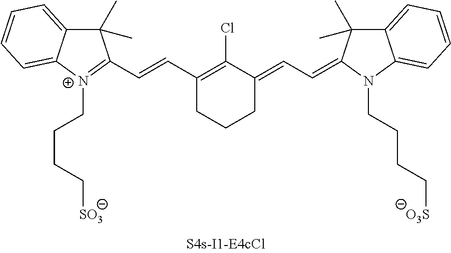 | excellent | excellent |
| 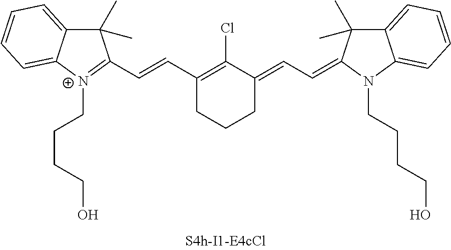 [SMILES: CC1(C)C(/C=C/C2=C(Cl)C(=C/C=C3/N(CCCCO)c4ccccc4C3(C)C)/CCC2)=[N+](CCCCO)c2ccccc21] | poor | poor |
| 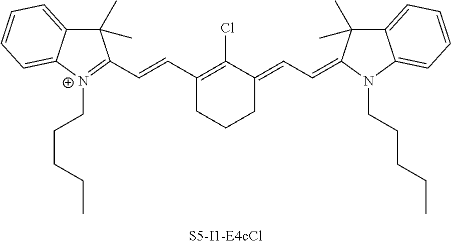 [SMILES: CCCCCN1/C(=C/C=C2\CCCC(/C=C/C3=[N+](CCCCC)c4ccccc4C3(C)C)=C2Cl)C(C)(C)c2ccccc21] | to be tested | to be tested |
| 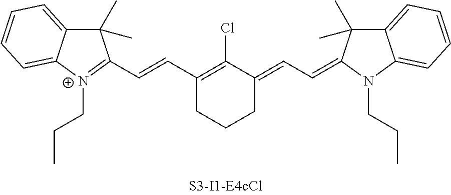 [SMILES: CCCN1/C(=C/C=C2\CCCC(/C=C/C3=[N+](CCC)c4ccccc4C3(C)C)=C2Cl)C(C)(C)c2ccccc21] | to be tested | to be tested |
| 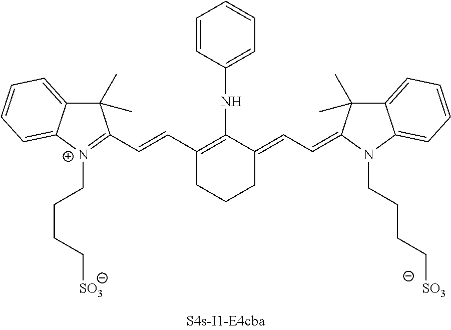 | excellent | poor |
| 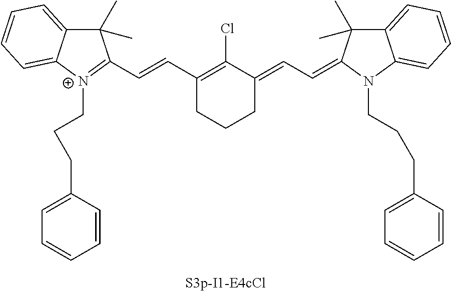 [SMILES: CC1(C)C(/C=C/C2=C(Cl)C(=C/C=C3/N(CCCc4ccccc4)c4ccccc4C3(C)C)/CCC2)=[N+](CCCc2ccccc2)c2ccccc21] | excellent | poor |
| 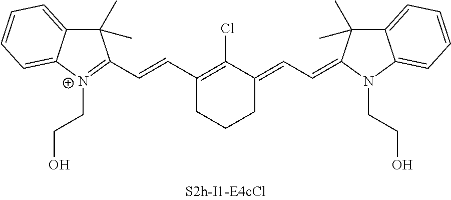 [SMILES: CC1(C)C(/C=C/C2=C(Cl)C(=C/C=C3/N(CCO)c4ccccc4C3(C)C)/CCC2)=[N+](CCO)c2ccccc21] | excellent | low intensity |
| 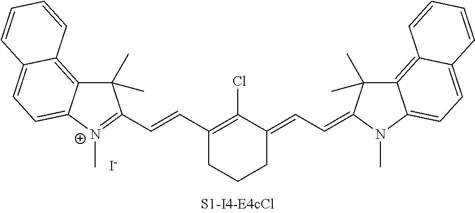 [SMILES: CN1/C(=C/C=C2\CCCC(/C=C/C3=[N+](C)c4ccc5ccccc5c4C3(C)C)=C2Cl)C(C)(C)c2c1ccc1ccccc21 ; [I-]] | | poor |
| 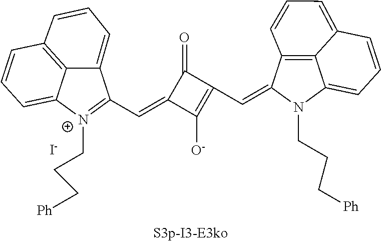 [SMILES: O=C1C(/C=c2\c3cccc4cccc(c43)n2CCCc2ccccc2)=C([O-])C/1=C/C1=[N+](CCCc2ccccc2)c2cccc3cccc1c23 ; [I-]] | | poor |
| 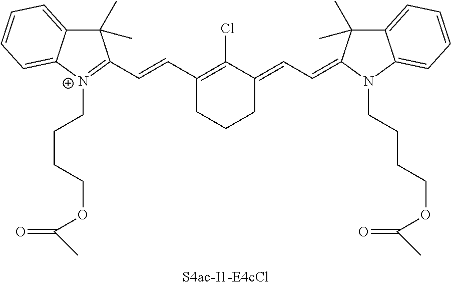 [SMILES: CC(=O)OCCCCN1/C(=C/C=C2\CCCC(/C=C/C3=[N+](CCCCOC(C)=O)c4ccccc4C3(C)C)=C2Cl)C(C)(C)c2ccccc21] | to be tested | to be tested |
| 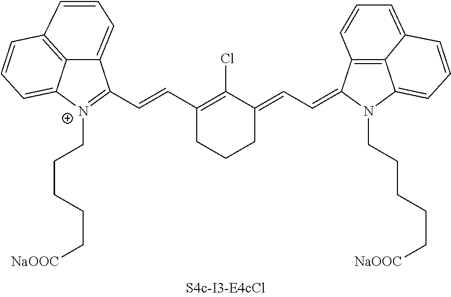 | poor | poor |
| 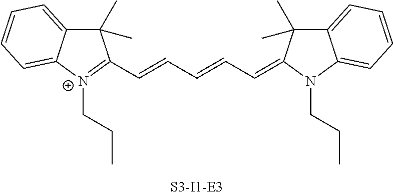 [SMILES: CCCN1/C(=C/C=C/C=C/C2=[N+](CCC)c3ccccc3C2(C)C)C(C)(C)c2ccccc21] | to be tested | to be tested |
| 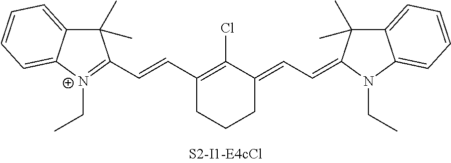 [SMILES: CCN1/C(=C/C=C2\CCCC(/C=C/C3=[N+](CC)c4ccccc4C3(C)C)=C2Cl)C(C)(C)c2ccccc21] | to be tested | to be tested |

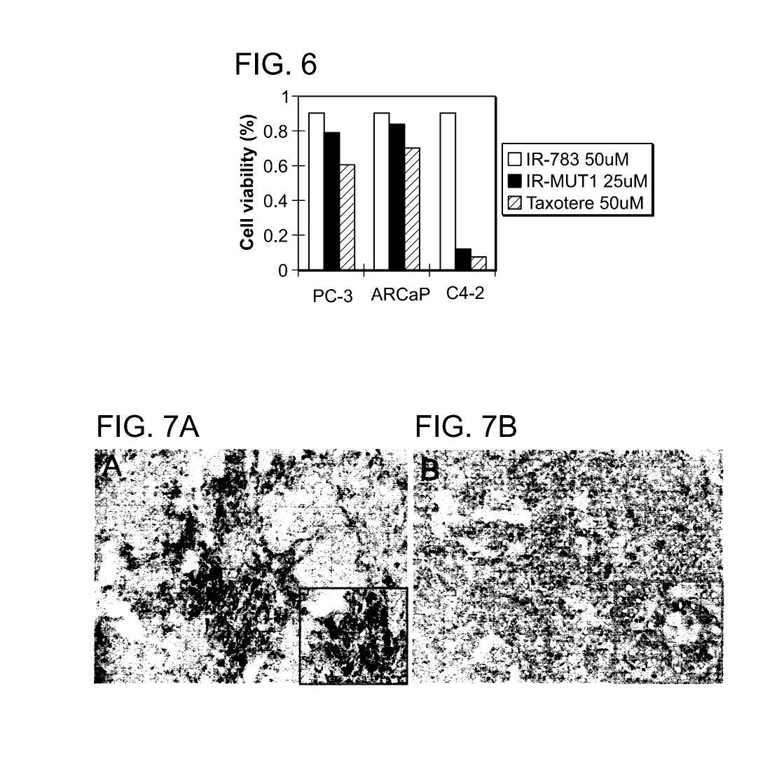

IR-783 (dye molecule only) is nontoxic. FIG. 6 shows that IR-MUT1 is toxic and can kill cancer cells, but is less toxic than the free nonconjugated drug, taxotere, when evaluated at day 2. This is expected since IR783 conjugated to docetaxel or paclitaxel, accumulation in cells require enzymatic activation, which releases the active taxotere or taxol component inside of the cells to exert cytotoxicity against the growth of cancer cells. This shows that targeted, sustained cancer therapy can be carried out with the ligand-drug conjugates described here.

Apoptosis

The ligand-drug conjugates of the invention show apoptosis of tumor cells. FIGS. 7A-7B are an assessment of apoptosis of mouse tumor tissue (A) T24 human bladder tumor xenograft nude mouse treated with IR-MUT1. (B) Control T24 human bladder xenograft mouse without treatment. Cytodeath stain with M30 antibody showed clear apoptosis in the IR-MUT1 treated tumor in an athymic nude mouse. Shown is 10× of a frozen section of a T24 tumor with inset showing a magnification of 20×. Note: the dark deposits represent the apoptotic cells. In the control mouse, there is no evidence of apoptosis shown by the lack of M30 Cytodeath antibody staining in this tissue section (10× of the picture with a 20× of inset).

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

All patents and publications mentioned in the specification are indicative of the levels of skill of those skilled in the art to which the invention pertains. References cited herein are incorporated by reference herein in their entirety to indicate the state of the art, in some cases as of their filing date, and it is intended that this information can be employed herein, if needed, to exclude (for example, to disclaim) specific embodiments that are in the prior art. For example, when a compound is claimed, it should be understood that compounds known in the prior art, including certain compounds disclosed in the references disclosed herein (particularly in referenced patent documents), are not intended to be included in the claim.

When a group of substituents is disclosed herein, it is understood that all individual members of those groups and all subgroups, including any isomers and enantiomers of the group members, and classes of compounds that can be formed using the substituents are disclosed separately. When a compound is claimed, it should be understood that compounds known in the art including the compounds disclosed in the references disclosed herein are not intended to be included. When a Markush group or other grouping is used herein, all individual members of the group and all combinations and subcombinations possible of the group and other groups presented are intended to be individually included in the disclosure.

Every formulation or combination of components described or exemplified can be used to practice the invention, unless otherwise stated. Specific names of compounds are intended to be exemplary, as it is known that one of ordinary skill in the art can name the same compounds differently. When a compound is described herein such that a particular isomer or enantiomer of the compound is not specified, for example, in a formula or in a chemical name, that description is intended to include each isomer and enantiomer of the compound described individual or in any combination. One of ordinary skill in the art will appreciate that methods, drug compounds, starting materials, synthetic methods, and conjugate components other than those specifically exemplified can be employed in the practice of the invention without resort to undue experimentation. All art-known functional equivalents, of any such methods, drug compounds, starting materials, synthetic methods, and conjugate components are intended to be included in this invention. Whenever a range is given in the specification, for example a composition range, all intermediate ranges and subranges, as well as all individual values included in the ranges given are intended to be included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In general the terms and phrases used herein have their art-recognized meaning, which can be found by reference to standard texts, journal references and contexts known to those skilled in the art. The following definitions are provided to clarify their specific use in the context of the invention.

One skilled in the art readily appreciates that the present invention is well adapted to carry out the objects and obtain the ends and advantages mentioned, as well as those inherent in the present invention. The methods, components, materials and dimensions described herein as currently representative of preferred embodiments are provided as examples and are not intended as limitations on the scope of the invention. Changes therein and other uses which are encompassed within the spirit of the invention will occur to those skilled in the art, are included within the scope of the claims.

Although the description herein contains certain specific information and examples, these should not be construed as limiting the scope of the invention, but as merely providing illustrations of some of the embodiments of the invention. Thus, additional embodiments are within the scope of the invention.

The exact formulation, route of administration and dosage can be chosen by the individual physician in view of the patient's condition (see e.g. Fingl et. al., in The Pharmacological Basis of Therapeutics, 1975, Ch. 1 p. 1). Routes of administration and dosages known in the art may be found in Comprehensive Medicinal Chemistry, Volume 5, Hansch, C. Pergamon Press, 1990.

It should be noted that the attending physician would know how to and when to terminate, interrupt, or adjust administration due to undesired toxicity, or to organ dysfunctions, or to other adverse effects. Conversely, the attending physician would also know to adjust treatment to higher levels if the clinical response were not adequate (precluding toxicity). The magnitude of an administered dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above also may be used in veterinary medicine.

Depending on the specific conditions being treated and the targeting method selected, such agents may be formulated and administered systemically or locally. Techniques for formulation and administration may be found in Alfonso and Gennaro (1995). Suitable routes may include, for example, oral, rectal, transdermal, vaginal, transmucosal, or intestinal administration; parenteral delivery, including intramuscular, subcutaneous, or intramedullary injections, as well as intrathecal, intravenous, or intraperitoneal injections.

For injection, the agents of the invention may be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular those formulated as solutions, may be administered parenterally, such as by intravenous injection. Appropriate compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve the intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions, including those formulated for delayed release or only to be released when the pharmaceutical reaches the small or large intestine.

The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

1. A conjugate compound comprising: a targeting ligand comprising a side chain portion, a small molecule anti-cancer; drug, and a linker comprising a succinic ester, wherein the linker connects the targeting ligand to the anti-cancer drug; the targeting ligand comprises two indole groups each comprising a nitrogen atom, and a polyen that (a) contains an aromatic or non-aromatic ring and (b) attaches one of the two indole groups to the other; and the side chain portion extends from the nitrogen atom of the indole group (I) as represented by the following formula: 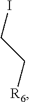
wherein R₆ is selected from the group consisting of: OH; NH₂; SH; COOH; C₁-C₁₅ alkyl and is optionally substituted with one or more nitrogen-containing groups, oxygen-containing groups, sulfur-containing groups or halogen atoms; alkoxy and is optionally substituted with one or more nitrogen-containing groups, oxygen-containing groups, sulfur-containing groups or halogen atoms; aryl and is optionally substituted by one or more heteroatoms or substituents; aromatic ring and is optionally substituted by one or more heteroatoms or substituents; non-aromatic ring and is optionally substituted by one or more heteroatoms or substituents; oxy; carbonyl; alkenyl; nitro; and amino.

2.-3. (canceled)

4. The compound of claim 1, wherein the targeting ligand comprises a conjugation amenable functional group.

5. The compound of claim 4, wherein the conjugation amenable functional group is selected from the group consisting of OH, NH₂, SH, and COOH.

6. The compound of claim 1, wherein an indole group and the polyen are represented by the following formula: 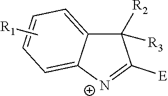
wherein E represents the polyen and R₁, R₂, and R₃ are each independently selected from the group consisting of: OH; NH₂; SH; COOH; H; C₁-C₁₅ alkyl and is optionally substituted with one or more nitrogen-containing groups, oxygen-containing groups, sulfur-containing groups or halogen atoms; alkoxy and is optionally substituted with one or more nitrogen-containing groups, oxygen-containing groups, sulfur-containing groups or halogen atoms; aryl and is optionally substituted by one or more heteroatoms or substituents; aromatic ring and is optionally substituted by one or more heteroatoms or substituents; non-aromatic ring and is optionally substituted by one or more heteroatoms or substituents; oxy; carbonyl; alkenyl; nitro; and amino.

7. The compound of claim 1, wherein the polyen is a polyen substituted with a substituent selected from the group consisting of OH, NH₂, SH, and COOH.

8. The compound of claim 1, wherein the aromatic or non-aromatic ring is substituted by one or more heteroatoms or substituents, wherein the one or more substituents is selected from the group consisting of OH, NH₂, SH, and COOH.

9. (canceled)

10. The compound of claim 1, wherein at least one of the two indole groups is selected from the group consisting of:

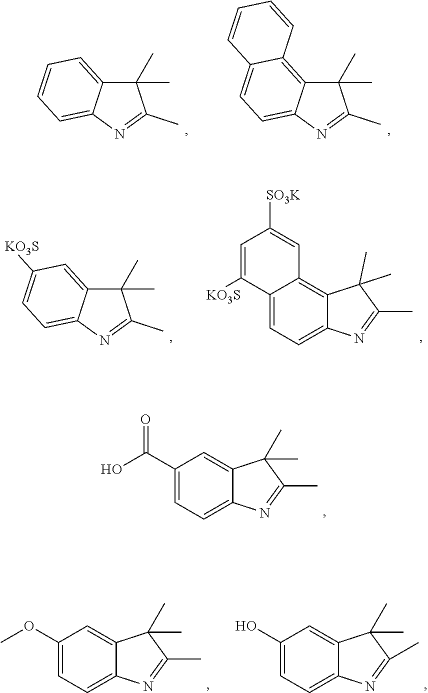

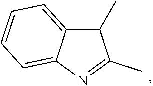

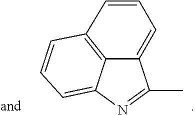

11. The compound of claim 1, wherein the polyen that attaches one of the two indole groups (I) to the other (I) is represented by:

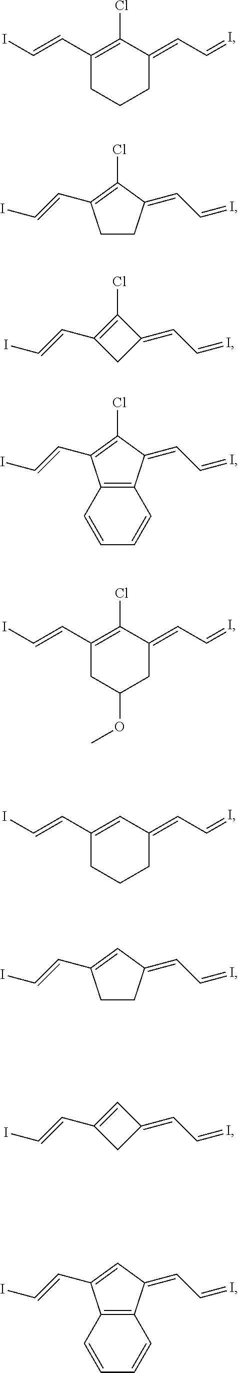

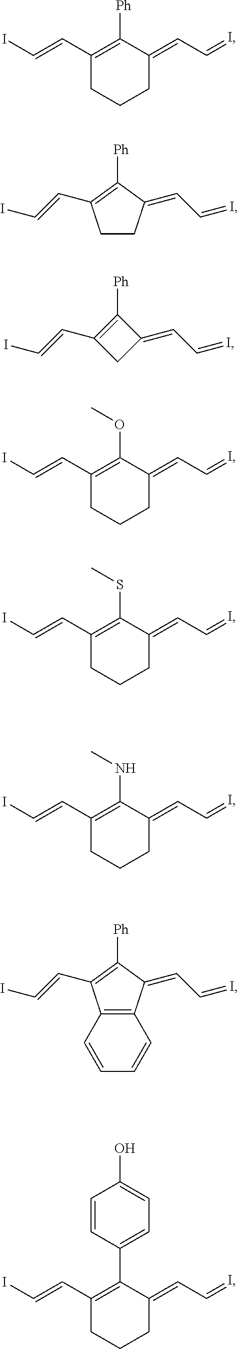

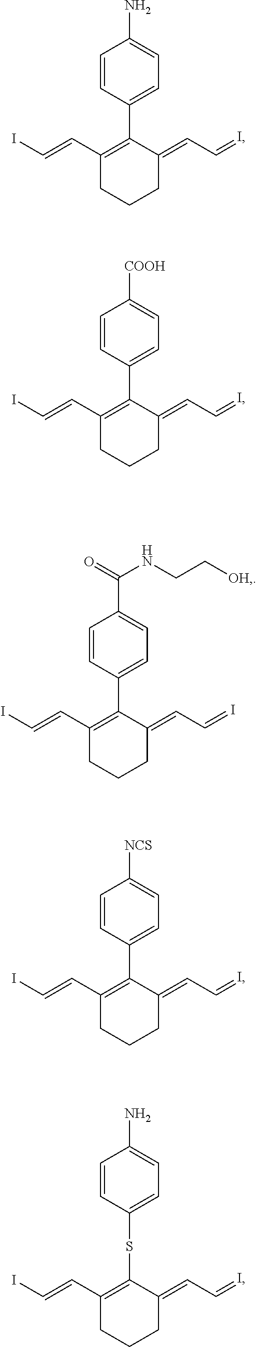

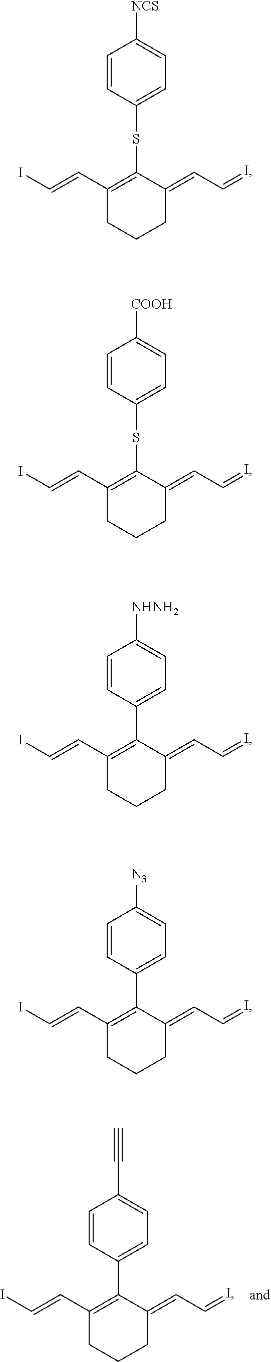

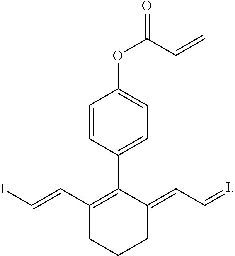

12. The compound of claim 1, wherein the side chain portion extends from an indole group (I) as represented by one of the following formulas:

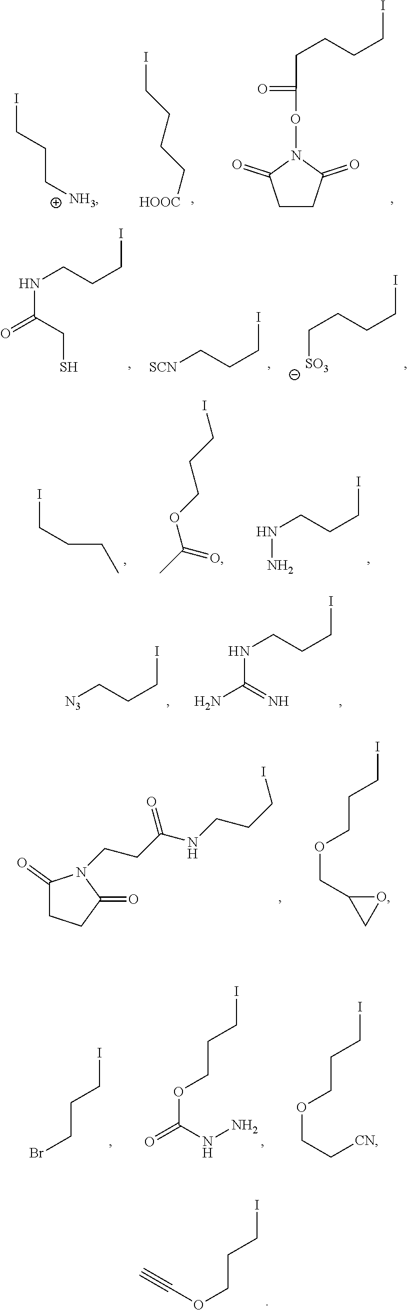

13.-14. (canceled)

15. The compound of claim 1, wherein the targeting ligand incorporated into the conjugate is a cyanine dye.

16. The compound of claim 15, wherein the cyanine dye incorporated into the conjugate as the targeting ligand is 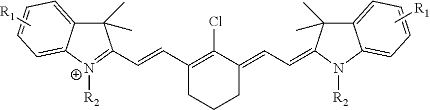
wherein R₁ and R₂ are each independently selected from the group consisting of: H; C₁-C₁₅ alkyl and is optionally substituted with one or more nitrogen-containing groups, oxygen-containing groups, sulfur-containing or halogen atoms; alkoxy and is optionally substituted with one or more nitrogen-containing groups, oxygen-containing groups, sulfur-containing or halogen atoms; aryl and is optionally substituted by one or more heteroatoms or substituents; aromatic ring and is optionally substituted by one or more heteroatoms or substituents; non-aromatic ring and is optionally substituted by one or more heteroatoms or substituents; oxy; carbonyl; alkenyl; nitro; and amino.

17. The compound of claim 15, wherein the cyanine dye incorporated into the conjugate as the targeting ligand is selected from the group consisting of:

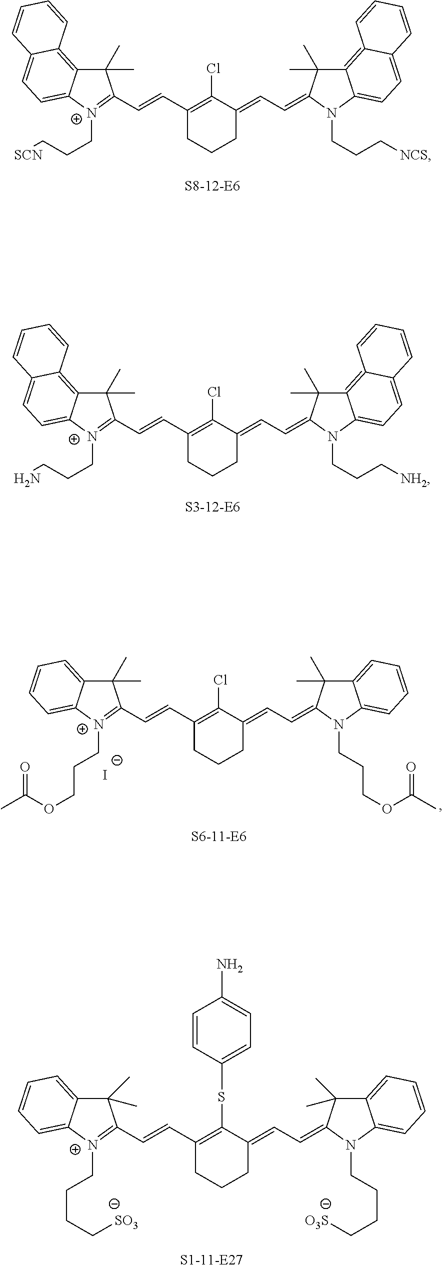

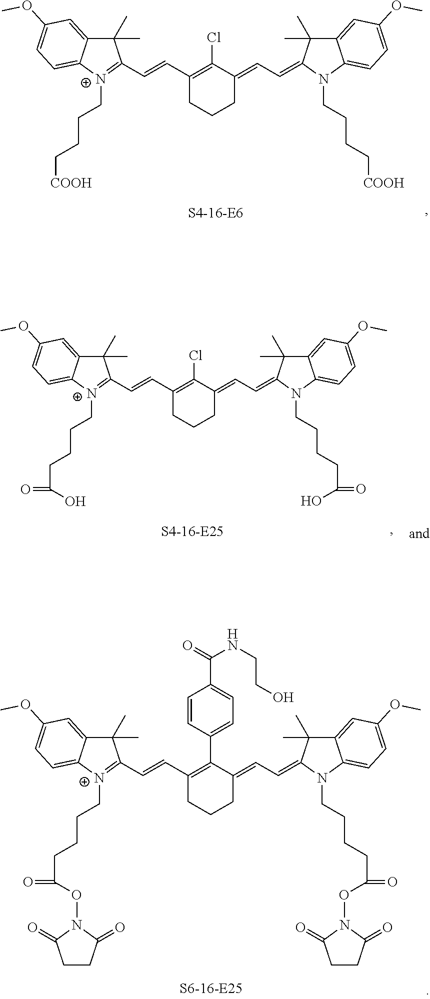

18. The compound of claim 1, wherein the targeting ligand incorporated into the conjugate is IR-783 or a derivative thereof.

19. (canceled)

20. The compound of claim 1, wherein the targeting ligand is a dye having wavelength of maximum fluorescence emission greater than 700 nm.

21. (canceled)

22. The compound of claim 1, wherein the small molecule anti-cancer drug is capable of targeting cell growth, survival, angiogenesis, adhesion, migration, invasion, metastasis, cell cycle progression and/or cell differentiation or is a bisphosphonate drug for metastatic bone cancer treatment.

23. The compound of claim 22, further comprising a ligand capable of recognizing tumor stroma, tumor cells, and/or matrices in a tumor microenvironment.

24. The compound of claim 23, wherein the ligand capable of recognizing tumor stroma, tumor cells, and/or matrices in the tumor microenvironment is selected from the group consisting of an RGD peptide that recognizes cell surface integrin receptors, a growth factor that recognizes cell surface growth factor receptors, or a peptide or small molecule substrate that recognizes functional cell surface plasminogen activator, bombesin, bradykinin or prostate-specific membrane antigen receptors.

25. The compound of claim 22, wherein the small molecule anti-cancer drug incorporated into the conjugate is selected from the group consisting of: aminoglutethimide, asparaginase, bleomycin, busulfan, carboplatin, carmustine (BCNU), chlorambucil, cisplatin (cis-DDP), cyclophosphamide, cytarabine HCl, dacarbazine, dactinomycin, daunorubicin HCl, doxorubicin HCl, estramustine phosphate sodium, etoposide (VP-16), floxuridine, fluorouracil (5-FU), flutamide, hydroxyurea, hydroxycarbamide, ifosfamide, leuprolide acetate, lomustine (CCNU), mechlorethamine HCl, melphatan, mercaptopurine, mesna, methotrexate (MTX), mitomycin, mitotane (o.p′-DDD), mitoxantrone HCl, octreotide, plicamycin, procarbazine HCl, streptozocin, tamoxifen citrate, thioguanine, thiotepa, vinblastine sulfate, vincrinstine sulfate, amsacrine (m-AMSA), azacitidine, hexamethylmelamine (HMM), mitoguazone (methyl-GAG, methyl glyoxal bis-guanylhydrazone (MGBG)), pentostatin, semustine (methyl-CCNU), teniposide (VM-26), paclitaxel, docetaxel, taxane, vindesine, and sulfate.

26. The compound of claim 1, wherein the small molecule anti-cancer drug incorporated into the conjugate is paclitaxel or docetaxel.

27. (canceled)

28. The compound of claim 22, wherein the bisphosphonate drug incorporated into the conjugate is zoledronate or pamidronate.

29. (canceled)

30. The compound of claim 1, wherein the compound is S4s-I1-E4cCI-Suc-docetaxel or S4s-I1-E4cCI-Suc-paclitaxel.

31.-75. (canceled)

76. The compound of claim 18, wherein the IR-783 derivative incorporated into the conjugate is selected from the group consisting of:

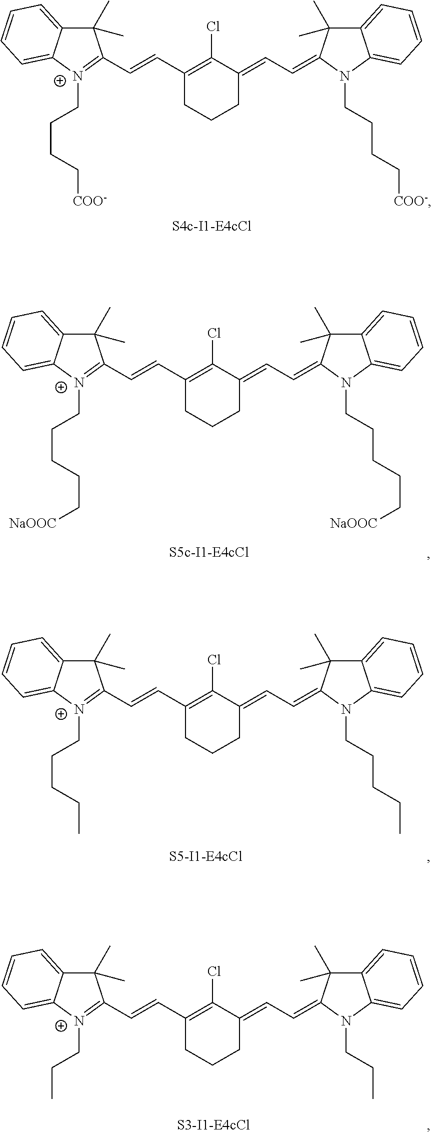

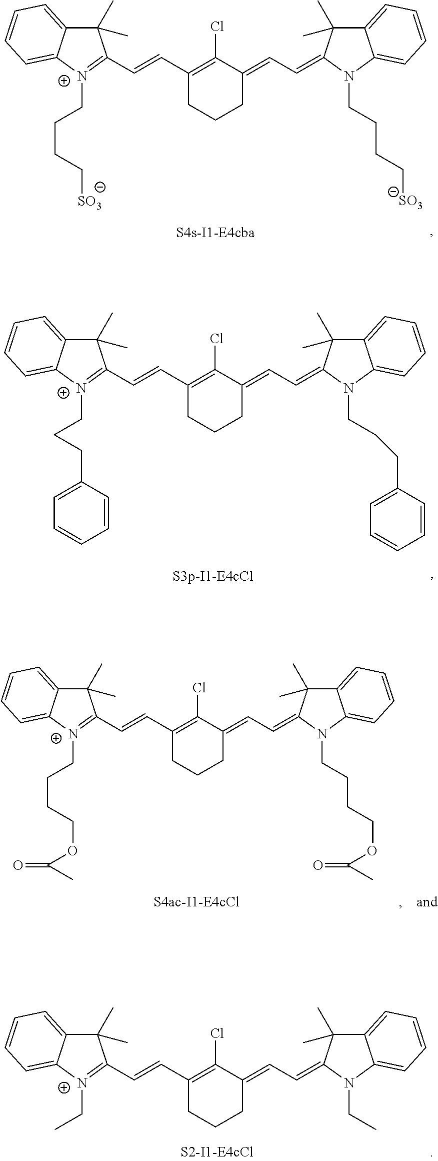

77. The conjugate of claim 1, wherein the small molecule anti-cancer drug does not selectively bind a tumor.
